SEQUENCE LISTING (23 sequences)

SEQ ID NO: 1 - protein, 8 aa, synthetic construct
DYKDDDDK

SEQ ID NO: 2 - protein, 662 aa, Homo sapiens
MSHVAVENALGLDQQFAGLDLNSSDNQSGGSTASKGRYIPPHLRNREATKGFYDKDSSGWSSSKDKDAYSSFGSRSDSRGKSSFFSDRGSGSRGRFDDRGRSDYDGIGSRGDRSGFGKFERGGNSRWCDKSDEDDWSKPLPPSERLEQELFSGGNTGINFEKYDDIPVEATGNNCPPHIESFSDVEMGEIIMGNIELTRYTRPTPVQKHAIPIIKEKRDLMACAQTGSGKTAAFLLPILSQIYSDGPGEALRAMKENGRYGRRKQYPISLVLAPTRELAVQIYEEARKFSYRSRVRPCVVYGGADIGQQIRDLERGCHLLVATPGRLVDMMERGKIGLDFCKYLVLDEADRMLDMGFEPQIRRIVEQDTMPPKGVRHTMMFSATFPKEIQMLARDFLDEYIFLAVGRVGSTSENITQKVVWVEESDKRSFLLDLLNATGKDSLTLVFVETKKGADSLEDFLYHEGYACTSIHGDRSQRDREEALHQFRSGKSPILVATAVAARGLDISNVKHVINFDLPSDIEEYVHRIGRTGRVGNLGLATSFFNERNINITKDLLDLLVEAKQEVPSWLENMAYEHHYKGSSRGRSKSSRFSGGFGARDYRQSSGASSSSFSSSRASSSRSGGGGHGSSRGFGGGGYGGFYNSDGYGGNYNSQGVDWWGN

SEQ ID NO: 3 - protein, 660 aa, Homo sapiens
MSHVVVKNDPELDQQLANLDLNSEKQSGGASTASKGRYIPPHLRNREASKGFHDKDSSGWSCSKDKDAYSSFGSRDSRGKPGYFSERGSGSRGRFDDRGRSDYDGIGNRERPGFGRFERSGHSRWCDKSVEDDWSKPLPPSERLEQELFSGGNTGINFEKYDDIPVEATGSNCPPHIENFSDIDMGEIIMGNIELTRYTRPTPVQKHAIPIIKGKRDLMACAQTGSGKTAAFLLPILSQIYTDGPGEALKAVKENGRYGRRKQYPISLVLAPTRELAVQIYEEARKFSYRSRVRPCVVYGGADIGQQIRDLERGCHLLVATPGRLVDMMERGKIGLDFCKYLVLDEADRMLDMGFEPQIRRIVEQDTMPPKGVRHTMMFSATFPKEIQMLARDFLDEYIFLAVGRVGSTSENITQKVVWVEDLDKRSFLLDILGATGSDSLTLVFVETKKGADSLEDFLYHEGYACTSIHGDRSQRDREEALHQFRSGKSPILVATAVAARGLDISNVRHVINFDLPSDIEEYVHRIGRTGRVGNLGLATSFFNEKNMNITKDLLDLLVEAKQEVPSWLENMAYEHHYKGGSRGRSKSNRFSGGFGARDYRQSSGSSSSGFGASRGSSSRSGGGGYGNSRGFGGGGYGGFYNSDGYGGNYNSQGVDWWGN

SEQ ID NO: 4 - protein, 662 aa, Mus musculus
MSHVAVENALGLDQQFAGLDLNSSDNQSGGSTASKGRYIPPHLRNREATKGFYDKDSSGWSSSKDKDAYSSFGSRGDSRGKSSFFGDRGSGSRGRFDDRGRGDYDGIGGRGDRSGFGKFERGGNSRWCDKSDEDDWSKPLPPSERLEQELFSGGNTGINFEKYDDIPVEATGNNCPPHIESFSDVEMGEIIMGNIELTRYTRPTPVQKHAIPIIKEKRDLMACAQTGSGKTAAFLLPILSQIYADGPGEALRAMKENGRYGRRKQYPISLVLAPTRELAVQIYEEARKFSYRSRVRPCVVYGGAEIGQQIRDLERGCHLLVATPGRLVDMMERGKIGLDFCKYLVLDEADRMLDMGFEPQIRRIVEQDTMPPKGVRHTMMFSATFPKEIQMLARDFLDEYIFLAVGRVGSTSENITQKVVWVEEIDKRSFLLDLLNATGKDSLTLVFVETKKGADSLEDFLYHEGYACTSIHGDRSQRDREEALHQFRSGKSPILVATAVAARGLDISNVKHVINFDLPSDIEEYVHRIGRTGRVGNLGLATSFFNERNINITKDLLDLLVEAKQEVPSWLENMAFEHHYKGSSRGRSKSSRFSGGFGARDYRQSSGASSSSFSSSRASSSRSGGGGHGGSRGFGGGGYGGFYNSDGYGGNYNSQGVDWWGN

SEQ ID NO: 5 - protein, 658 aa, Mus musculus
MSQVAAESTAGLDQQFVGLDLKSSDNQNGGGNTESKGRYIPPHLRNRETSKGVCDKDSSGWSCSKDKDAYSSFGSRDSRGKPNYFSDRGSGSRGRFDDHGRNDYDGIGGRDRTGFGKFERSGHSRWSDRSDEDDWSKPLPPSERLEQELFSGGNTGINFEKYDDIPVEATGNNCPPHIENFSDIEMGEIIMGNIELTRYTRPTPVQKHAIPIIKEKRDLMACAQTGSGKTAAFLLPILSQIYTDGPGEALKAMKENGRYGRRKQYPISLVLAPTRELAVQIYEEARKFSYRSRVRPCVVYGGADTVQQIRDLERGCHLLVATPGRLVDMMERGKIGLDFCKYLVLDEADRMLDMGFEPQIRRIVEQDTMPPKGVRHTMMFSATFPKEIQMLARDFLDEYIFLAVGRVGSTSENITQKVVWVEELDKRSFLLDLLNATGKDSLTLVFVETKKGADSLENFLFQERYACTSIHGDRSQKDREEALHQFRSGRKPILVATAVAARGLDISNVKHVINFDLPSDIEEYVHRIGRTGRVGNLGLATSFFNERNLNITKDLLDLLVEAKQEVPSWLESMAYEHHYKGSSRGRSKSRFSGGFGARDYRQSSGSANAGFNSNRANSSRSSGSSHNRGFGGGGYGGFYNNDGYGGNYNSQAVDWWGN

SEQ ID NO: 6 - DNA, 204 bp, Synthetic construct
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 7 - DNA, 1653 bp, Synthetic construct
  source 1..1653: mol_type = other DNA
atggaagacgccaaaaacataaagaaaggcccggcgccattctatccgctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaactcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggataccaggtggcccccgctgaattggagtcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaa

SEQ ID NO: 8 - DNA, 587 bp, Synthetic construct
  source 1..587: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacc

SEQ ID NO: 9 - DNA, 720 bp, Synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggatgagctgtacaagtga

SEQ ID NO: 10 - DNA, 399 bp, Synthetic construct
  source 1..399: mol_type = other DNA
atggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaa

SEQ ID NO: 11 - DNA, 28 bp, Synthetic construct
  source 1..28: mol_type = other DNA
ggtttagtgaaccgtcagatccgctagc

SEQ ID NO: 12 - DNA, 582 bp, Synthetic construct
  source 1..582: mol_type = other DNA
atttgggagagtgctgtgactcatgctggactctaacccacgagggtttctcagagtcagcagctgggggatgaagaagtgaaaagtgactggcaggaaattctgcaagcaaggaaagggaaagagaaatgaactggtgcaggtctgcgggaagagaatgaggctggatcctcaaaatcacaggaggaagcaggcccagacctcagaggcagaaagagaaagaaaccagagcttagagtcaggaggaggaaaccagaccccggagccacaaggagagggctggatccccggctcagagggaagagtgtcgccgcctctgcctgcgtagccccggccatggctctgtagcctcgacccctttgtgcccccggcccgtctccgcgctcaccacgcctgcgctctccgctcccaccttctttcttcagccgaggccgccgccgcctctccttgctgcagccatggagtcttccactttcgccttggtgcctgtcttcgcccacctgagcatcctccagagcctcgtgccagctgctggtgcagcctctcctgttgccatcagtgcccagcacctgtgctacagcc

SEQ ID NO: 13 - DNA, 165 bp, Synthetic construct
  source 1..165: mol_type = other DNA
tggacacataagaggctgcgtataggcgcgagagcccctttcctcagctgccgccaaggtgctcggtccttccgaggaagctaaggctgcgttggggtgaggccctcacttcatccggcgactagcaccgcgtccggcagcgccagccctacactcgcccgcgcc

SEQ ID NO: 14 - DNA, 185 bp, Synthetic construct
  source 1..185: mol_type = other DNA
tcgcaccccgcggcccgccccccgccgccaccctcgcagatccgtgctttttcccctttgcttctctcccgtactgggtcagtcctgtccgcgctcgcgcgtcggtttgcgggtgtgcgcaggcgcggcaggggccattagccctttgggtgggcggtggagcccgggagcgcgcgggcgagacc

SEQ ID NO: 15 - DNA, 285 bp, Synthetic construct
  source 1..285: mol_type = other DNA
ttaagtcacgtgacatgaggagaggtgggcgggtacctggaggaagctcgcggcgtcggtggcggtggcgcgcggcggccgctgagaccggggctttgagtcgcaccccgcggcccgccccccgccgccaccctcgcagatccgtgctttttcccctttgcttctctcccgtactgggtcagtcctgtccgcgctcgcgcgtcggtttgcgggtgtgcgcaggcgcggcaggggccattagccctttgggtgggcggtggagcccgggagcgcgcgggcgagacc

SEQ ID NO: 16 - DNA, 201 bp, Synthetic construct
  source 1..201: mol_type = other DNA
gcgagggggcgtagccggagctacggcaccaaggctccgcccccaccctgcctgccccctcgctggagcaacgcaagcaggaggcgggggagtcggaggaggtggcggcgctggagctcctcccggggaccagcgacccggggagcgagcacgtcgctccgcaccgctcttcctccagccgctgagccgtcccttctcgcc

SEQ ID NO: 17 - DNA, 197 bp, Synthetic construct
  source 1..197: mol_type = other DNA
agttttcctcagctttgggtggtggccgctgccgggcatcggcttccagtccgcggagggcgaggcggcgtggacagcggccccggcacccagcgccccgccgcccgcaagccgcgcgcccgtccgccgcgccccgagcccgccgcttcctatctcagcgccctgccgccgccgccgcggcccagcgagcggccctg

SEQ ID NO: 18 - DNA, 511 bp, Synthetic construct
  source 1..511: mol_type = other DNA
gacgtcggcccgccggcgccccaccagctccgcgcgggcccgggttggccaccgccgggcccccgcccctcccccggcggtgtcccggccggaaccgatcgtggctggtttgagctggtgcgtctccatggcgacccgccggtgctataagtagggagcggcgtgccgtggggctttgtcagtccctcctgtagccgccgccgccgccgcccgccgcccctctgccagcagctccggcgccacctcgggccggcgtctccggcgggcgggagccaggcgctgacgggcgcggcgggggcggccgagcgctcctgcggctgcgactcaggctccggcgtctgcgcttccccatggggctggcctgcggcgcctgggcgctctgagattgtcactgctgttccaagggcacacgcagagggatttggaattcctggagagttgcctttgtgagaagctggaaatatttctttcaattccatctcttagttttccataggaacatcaagaaatc

SEQ ID NO: 19 - DNA, 279 bp, Synthetic construct
  source 1..279: mol_type = other DNA
gaggggctgggagccgcggcggggcggtgcgagggcgggccggggccggttccgcgcgcagggattttaaatgtcccgctctgagccgggcgcaggagcagccggcgcggccgccagcgcggtgtagggggcaggcgcggatcccgccaccgccgcgcgctcggcccgccgactcccggcgccgccgccgccactgccgtcgccgccgccgcctgccgggactggagcgcgccgtccgccgcggacaagaccctggcctcaggccggagcagccccatc

SEQ ID NO: 20 - DNA, 20 bp, Homo sapiens
  source 1..20: mol_type = genomic DNA
cgtggacggagtgattacga

SEQ ID NO: 21 - DNA, 20 bp, Homo sapiens
  source 1..20: mol_type = genomic DNA
cggagtgattacgatggcat

SEQ ID NO: 22 - DNA, 20 bp, Homo sapiens
  source 1..20: mol_type = genomic DNA
gcaaatatgtgaaggagctc

SEQ ID NO: 23 - DNA, 20 bp, Synthetic construct
  source 1..20: mol_type = other DNA
gtgtagttcgaccattcgtg

(19) United States
(12) Patent Application Publication
(10) Pub. No.: US 2025/0207173 A1
(43) Pub. Date: Jun. 26, 2025

(54) REPORTER CELL FOR ASSESSING DDX3 HELICASE ACTIVITY

(71) Applicants: Nanyang Technological University, Singapore (SG); Singapore Health Services Pte Ltd, Singapore (SG)

(72) Inventors: Navin Kumar VERMA, Singapore (SG); Nicholas Francis GRIGOROPOULOS, Singapore (SG); Zhi Sheng POH, Singapore (SG)

(21) Appl. No.: 18/987,810

(22) Filed: Dec. 19, 2024

(30) Foreign Application Priority Data
Dec. 22, 2023 (SG) 10202303615S

(51) Int. Cl. C12Q 1/34; C12N 5/071; C12Q 1/26; G01N 21/76; G01N 33/50

(52) U.S. Cl. CPC C12Q 1/34; C12N 5/0686; C12Q 1/26; G01N 21/76; G01N 33/5023; C12N 2510/00; G01N 2333/90241; G01N 2333/914

(57) ABSTRACT

The present invention relates generally to the field of cell-based methods for assaying DDX3 helicase activity. In particular, various embodiments relate to an engineered reporter cell comprising a reporter expression construct capable of interrogating DDX3 helicase activity. Moreover, various embodiments also relate to methods, uses and systems applying the engineered reporter cell.

### CROSS-REFERENCE TO RELATED APPLICATION

This application claims the benefit of priority of Singapore Patent application No. 10202303615S filed 22 Dec. 2023, the content of which is hereby incorporated by reference in its entirety for all purposes.

### REFERENCE TO AN ELECTRONIC SEQUENCE LISTING

The contents of the electronic sequence listing (690148_629_SEQUENCE_LISTING.xml; Size: 29,491 bytes; and Date of Creation: Oct. 29, 2024) are herein incorporated by reference in their entirety.

### FIELD OF THE INVENTION

Various embodiments relate generally to the field of cell-based methods for assaying DDX3 helicase activity. In particular, various embodiments relate to an engineered reporter cell comprising a reporter expression construct capable of interrogating DDX3 helicase activity. Moreover, various embodiments also relate to methods, uses and systems applying the engineered reporter cell.

### BACKGROUND

DDX3 is a member of the highly conserved family of DEAD-box RNA helicases involved in the unwinding of RNA duplexes and mRNA translation [1]. Humans express two homologs of the DDX3 protein-DDX3X and DDX3Y. The DDX3X protein is encoded by the DDX3X gene on p11.3-11.23 of the X-chromosome and is ubiquitously expressed in all tissues [2]. The DDX3Y protein is encoded by the paralogous DDX3Y gene located in the non-recombining region of the Y chromosome. DDX3Y mRNA is present throughout all the tissues in males, but DDX3Y protein expression is restricted to the testis and plays an essential role in spermatogenesis and male fertility [3].

Both DDX3X and DDX3Y share ˜92% similarity in amino acid sequences and play an indispensable and often compensatory roles in various cellular processes, including pre-mRNA splicing, mRNA stability and translation, as well as in signal transduction such as the Wnt/β catenin and NF-κB pathways [4]. In addition, they regulate a wide range of biological processes, including cell adhesion, cell cycle, and cellular stress responses [4,5]. Germline inheritance of pathogenic DDX3X mutations hinder neurodevelopment, accounting for ˜1-3% cases with intellectual disability [6]. In cancer, DDX3X can function as a tumour suppressor or as an oncogene depending on tumour type [4] For example, DDX3X is overexpressed in breast, colorectal, lung, medulloblastoma, and prostate cancer [4,7]; whereas somaticaly mutated in medulloblastoma, melanoma, and non-Hodgkin lymphoma subtypes, such as natural killer/T-cell lymphoma (NKTCL), Burkitt's lymphoma (BL) and diffuse large B-cell lymphoma (DLBCL) [4,8-11].

In comparison to DDX3X, biological roles of DDX3Y are relatively less well understood. This could be mainly due to stage-specific expression of DDX3Y in the male testis during spermatogenesis that makes this protein challenging to isolate and characterize its molecular function. Nevertheless, due to their diverse and crucial roles in cell biology and multiple diseases, both DDX3X and DDX3Y are gaining increasing attention for research.

Although conventional helicase and ATPase assays have been utilized to determine the functions of DDX3X and DDX3Y and identify their pathogenic variants, these assays are complicated and time-consuming, and often technically challenging due to the requirement of protein synthesis and radiolabelling. Moreover, traditional assays are performed in cell-free systems which cannot address biologically relevant questions involving multiple cell signalling and molecular factors.

Therefore, there is still a need in the art for the provision of a cell-based model and method for assessing the activity of DDX3 helicases, such as DDX3X and DDX3Y, and their homologues, paralogues, and mutational variants to address the drawbacks of existing approaches.

### SUMMARY

In one aspect, there is provided a reporter cell for assessing the activity of a DDX3 helicase, comprising a circular DNA molecule comprising a reporter expression construct comprising a reporter gene operably linked to one or more regulatory elements capable of controlling expression of the reporter gene; and a nucleotide sequence encoding a 5′ untranslated region (5′-UTR) positioned upstream of the reporter gene, wherein a secondary structure of the 5′-UTR is capable of being unwound by a functionally active DDX3 helicase to facilitate the expression of the reporter gene and emission of a detectable signal, wherein the reporter cell lacks endogenous expression of the DDX3 helicase.

In various embodiments, the DDX3 helicase is a human DDX3X or DDX3Y helicase, or variants thereof.

In various embodiments, the reporter cell naturally or artificially lacks endogenous expression of the DDX3 helicase, more preferably the reporter cell is a DDX3 knock-out reporter cell.

In various embodiments, the reporter cell further comprises a second circular DNA molecule comprising a nucleotide sequence encoding the DDX3 helicase, and one or more regulatory elements capable of controlling exogenous or transient expression of the DDX3 helicase, preferably the second circular DNA molecule is an expression plasmid for transient and/or exogenous expression of the DDX3 helicase.

In various embodiments, the reporter gene is a luminescent reporter gene, preferably a gene encoding firefly luciferase (FLuc), a gene encoding renilla luciferase (Rluc), a gene encoding Cypridina Luciferase (Cluc), a gene encoding Gaussia Luciferase (Gluc), or a gene encoding NanoLuc Luciferase (NanoLuc), more preferably the reporter gene is a gene encoding firefly luciferase (FLuc).

In various embodiments, the nucleotide sequence encoding the 5′-UTR may be derived from a 5′-UTR of the RAC1 or DVL2 gene, preferably the nucleotide sequence encoding the 5′-UTR comprises or consists of a nucleotide sequence as set forth in any one of SEQ ID NO: 14 and 17 or variant thereof.

In various embodiments, the reporter expression construct further comprises an Internal Ribosome Entry Site (IRES) and a fluorescent marker gene, wherein the IRES is positioned downstream of the reporter gene, and the fluorescent marker gene is positioned downstream of the IRES.

In various embodiments, the reporter cell is a mammalian cell derived from a mammalian subject or a mammalian cell line, preferably the mammalian cell is a primary cell derived from a human subject or a human cell derived from a human cell line selected from Hela cells and human embryonic kidney (HEK-293) cells, diffuse large B-cell lymphoma cells (U2932), prostate epithelial cells (RWPE-1), Metastatic prostate carcinoma cells (LNCaP), more preferably the reporter cell is a human cell derived from an embryonic kidney 293 (HEK 293) cell line or derivatives thereof.

In a second aspect, there is provided a method for production of the reporter cell disclosed herein, the method comprising: providing a host cell that lacks endogenous expression of the DDX3 helicase; and introducing a circular DNA molecule into the host cell at suitable conditions to obtain the reporter cell, wherein the circular DNA molecule comprises a reporter expression construct comprising: a reporter gene operably linked to one or more regulatory element capable of controlling expression of the reporter gene; and a nucleotide sequence encoding a 5′ untranslated region (5′-UTR) positioned upstream of the reporter gene such that unwinding of a secondary structure of the 5′-UTR by a functionally active DDX3 helicase facilitates the expression of the reporter gene and emission of a detectable signal.

In various embodiments, the method further comprises introducing a second circular DNA molecule into the host cell at suitable conditions, wherein the second circular DNA molecule comprises a nucleotide sequence encoding a DDX3 helicase, and one or more regulatory elements capable of controlling exogenous or transient expression of the DDX3 helicase.

In a third aspect, there is provided a reporter cell obtained by the method of production disclosed herein.

In a fourth aspect, there is provided a cell culture comprising the reporter cell disclosed herein, and a cell culture medium suitable for expansion of the reporter cell.

In a fifth aspect, there is provided a use of a reporter cell disclosed herein for assessing the activity of DDX3 helicase.

In a sixth aspect, there is provided a method for assessing the activity of a DDX3 helicase, comprising: providing a reporter cell disclosed herein; culturing the reporter cell under conditions that allow expression of the reporter gene to produce a reporter gene product, wherein the DDX3 helicase is exogenously or transiently expressed in the reporter cell; and detecting a presence/absence, optionally quantity, of a detectable signal emitted by the reporter gene product to obtain a readout that is indicative of the activity of the DDX3 helicase.

In various embodiments, a presence, optional quantity, of the detectable signal emitted by the reporter gene product indicates that the exogenously or transiently expressed DDX3 helicase is functionally active and has unwound a secondary structure of the 5′-UTR facilitating expression of the reporter gene and emission of the detectable signal.

In various embodiments, the reporter gene is a luciferase reporter gene, and the detectable signal is a luminescence signal, and wherein a quantitative readout of the luminescence signal is proportional to the expression level of the luciferase reporter gene and correlates to the activity of the DDX3 helicase.

In a seventh aspect, there is provided a method for developing or identifying an agent capable of modulating the activity of DDX3 helicase, the method comprising: providing a reporter cell disclosed herein, wherein the reporter cell exogenously or transiently expresses a functionally active DDX3 helicase; administering a candidate agent to the reporter cell; measuring a detectable signal emitted by a reporter gene product; comparing the measured signal to a control, wherein the candidate agent is identified as capable of modulating the activity of DDX3 helicase, if the measured detectable signal of the reporter gene product has increased or decreased relative to the control.

In various embodiments, a decrease of the measured detectable signal of the reporter gene product relative to the control is indicative of the candidate agent having inhibitory activity of DDX3 helicase, or an increase of the measured detectable signal of the reporter gene product relative to the control is indicative of the candidate agent having stimulatory activity of DDX3 helicase.

### BRIEF DESCRIPTION OF THE DRAWINGS

Various embodiments will be better understood with reference to the detailed description when considered in conjunction with the non-limiting examples and the accompanying drawings.

FIG. 1A-1E shows the design and development of the novel ICD-helicase reporter cell system disclosed herein: FIG. 1A Schematic of luciferase reporter plasmid in which the complex 5′-UTR is placed upstream of the reporter gene; FIG. 1B Schematic of luciferase reporter plasmid in which the complex Rac1 5′-UTR is placed upstream of the firefly Luciferase (FLuc); FIG. 1C Schematic of luciferase reporter plasmid in which the complex DvL2 5′-UTR is placed upstream of FLuc. FIG. 1D Schematic of luciferase reporter plasmid in which the complex Rac1 5′-UTR is placed upstream of FLuc and the blasticidin gene under PGK promoter (PGK-BLAST) is placed downstream; FIG. 1E Schematic of luciferase reporter plasmid in which the complex DvL2 5′-UTR is placed upstream of FLuc and the PGK-BLAST is placed downstream. The Aequorea coerulescens green fluorescence protein (AcGFP) is added downstream as an internal control separated by an internal ribosome entry site (IRES). The schematic also provides RNA secondary structures of Rac1 5′-UTR (B) and DvL2 5′-UTR (C), created using ViennaRNA.

FIG. 2 shows flow-cytometry gating strategy adopted to determine ICD-Helicase reporter plasmid transfection efficiency in 293T cells within each sample for normalizing bioluminescence signals.

FIG. 3 shows 293T cells transfected with Rac1-FLuc or DvL2-FLuc reporter plasmids emitting luciferase signals in the presence of vehicle (DMSO) that were dose-dependently inhibited by a non-specific inhibitor RK-33 in the concentration range of 0.5 to 2 μM. Cells were treated with cycloheximide as an inhibitory control. Data represent three independent experiments and values in the bars are mean±SEM. *, p<0.05; **, p<0.01; ****, p<0.0001.

FIG. 4A shows the schematic and sequences of CRISPR/Cas9 knock out (KO) target sites for two different gRNAs against the DDX3X gene (sgRNA1 and sgRNA2). FIG. 4B shows representative immunoblots of control (sgCTL) and DDX3X KO (sgDDX3X) 293T cells probed for DDX3X and GAPDH (protein loading control). Blots are representative of at least three independent experiments.

FIG. 5 shows relative luciferase activities in wild-type control (sgCTL) and DDX3X KO (sgDDX3X) 293T Rac1-FLuc and DvL2-FLuc reporter cells. Firefly luciferase activity was determined by normalizing AcGFP⁺ levels within each sample before comparing against sgCTL firefly luciferase signals. Cells were treated with cycloheximide as an inhibitory control. Data represent three independent experiments and values in the bars are mean±SEM. ****, p<0.0001; ns, non-significant.

FIG. 6A shows the viability of reporter cells (sgCTL and sgDDX3X) treated with various RK-33 concentrations (0.5 μM, 1 μM, or 2 μM) that was determined by an MTS-based assay, normalized against the vehicle (DMSO). Staurosporine (STS) was used as a positive kill control. FIG. 6B shows representative brightfield images of wild-type (WT) and engineered sgCTL and sgDDX3X 293T cells. FIG. 6C shows apoptosis/cell death in control (sgCTL) and DDX3X knock-out (sgDDX3X) 293T cells that was determined using an Annexin V-FITC assay kit. Cells were treated with STS as a kill control. Bar chart represents mean±SEM of 3 independent experiments.

FIG. 7A-7B shows representative immunoblots of WT, sgCTL and sgDDX3X 293T cells from various clones and their relative luciferase activities: FIG. 7A Representative immunoblot of WT, sgCTL and sgDDX3X 293T cells from various clones. Cells were lysed and cellular lysates were analysed to determine the levels of DDX3X and GAPDH (loading control); and FIG. 7B Relative luciferase activity for the indicated treatment conditions. Firefly luciferase activity of reporter cells were normalized against AcGFP⁺ levels within each sample. Luminance signals were compared against sgCTL cells (Clone #1). Cells were treated with cycloheximide as an inhibitory control. Data represent mean±SEM of 3 independent experiments. ns, non-significant & ****, p<0.0001.

FIG. 8 shows the effect of RK-33 on the helicase activity of reporter cells. A total of 1.2×105 sgCTL or sgDDX3X reporter cells (Rac1-FLuc or DvL2-FLuc) were seeded into wells of a 24-well plate to allow cells to adhere for 24 h. Cells were pre-treated with increasing concentrations of a non-specific DDX3 inhibitor, RK-33 (0.5 μM, 1 μM, or 2 μM) for 3 h before transiently expressing the reporter plasmids into sgCTL and sgDDX3X cells for 16 h. Half of the cells were lysed in 1× cell culture lysis buffer (Promega #E1500) and bioluminescence was measured on a white bottom 96-well plate using luciferase assay system (Promega #E4030) by performing 2 sec measurement delay followed by a 10 sec measurement read. The remaining half of the cells were processed on flow-cytometry to determine the percentage of AcGFP⁺ cells, to account for variation in transfection efficiency. Graph shows firefly luciferase activity of reporter cells, normalized against AcGFP⁺ levels within each sample before comparing against sgCTL firefly luciferase activity. Cycloheximide was used as a positive control for translational inhibition. Data represent three independent experiments and values in the bars are mean±SEM. *, p<0.05; ****, p<0.0001; ns, non-significant.

FIG. 9 shows the schematic for interrogating helicase activities of wild-type (WT) and mutant DDX3X.

FIG. 10A shows representative immunoblots of sgCTL and sgDDX3X 293T cells that were transfected with either vector alone or WT-DDX3X or R475-DDX3X plasmid constructs, probed for DDX3X, Flag and GAPDH (loading control). FIG. 10B shows densitometry quantification of the DDX3X blot. Data represent three independent experiments.

FIG. 11A-11B shows relative luciferase activities of wild-type and mutant DDX3X using Rac1-FLuc and DvL2-FLuc reporter cells. Control (sgCTL) and DDX3X KO (sgDDX3X) 293T cells were transfected with plasmid vector alone, WT-DDX3X, or R475C-DDX3X plasmids. Relative luciferase activities of reporter cells with FIG. 11A Rac1-FLuc or FIG. 11B DvL2-FLuc were determined by normalizing AcGFP⁺ levels within each sample before comparing against sgCTL firefly luciferase signals. Cells were treated with cycloheximide as an inhibitory control. Data represent three independent experiments and values in the bars are mean±SEM. ****, p<0.0001.

FIG. 12 shows an illustration of the mutational landscape of DDX3X in DLBCL patients based on previous studies [8]. The 12 helicase motifs of DDX3X that are involved in ATP binding and hydrolysis, RNA binding, and the linker between RNA and ATP binding sites are shown. CTE, C-terminal extension; NTE, N-terminal extension.

FIG. 13A shows representative immunoblots of control (sgCTL) and DDX3X KO (sgDDX3X) 293T cells transfected with Flag-tagged DDX3X mutant plasmids as indicated. Blot was probed for DDX3X and Flag to confirm exogenous expression of mutant DDX3X and GAPDH as a protein loading control. FIG. 13B shows relative densitometry quantification of Western blot bands in protein samples from sgDDX3X cells that were transfected with plasmids to express WT-DDX3X, Y200H, R296C, Q309R, F357V, S470N, R475, R534H, or P568L. Data represent mean±SEM of 3 independent experiments.

FIG. 14 shows relative luciferase signals of reporter 293T cells produced by WT or mutant DDX3X as indicated, determined by normalizing AcGFP⁺ levels within each sample before comparing against sgCTL firefly luciferase signals. Cells were treated with cycloheximide as an inhibitory control. Data represent three independent experiments and values in the bars are mean±SEM. ****, p <0.0001. ns, non-significant.

FIG. 15 shows puromycin incorporation assay of reporter cells that were presented in FIG. 14. Briefly, sgCTL and sgDDX3X 293T cells transfected with Flag-tagged WT or mutant DDX3X plasmids as indicated were subjected to puromycin incorporation assay, lysed and cellular lysates were analysed by Western immunoblotting. The blot was also probed for GAPDH and stained with Ponceau S to confirm equal protein loading. Data represent three independent experiments.

FIG. 16 shows the quantification of DDX3X helicase activity in DLBCL cells. U2932 DLBCL cells were electroporated with the reporter plasmids. Relative luciferase signals of Rac1-FLuc and DvL2-FLuc U2932 reporter cells expressing WT-DDX3X or mutant R475C-DDX3X, determined by normalizing AcGFP⁺ levels within each sample before comparing against sgCTL firefly luciferase activity. Cells were treated with cycloheximide as an inhibitory control. Values are mean±SEM of three independent experiments. ****, p<0.0001; ns, non-significant.

FIG. 17A-17B shows the amino acid sequence alignment between human DDX3X and DDX3Y. FIG. 17A An illustration of DDX3X and DDX3Y amino acid sequence alignment highlighting identical and mismatched residues. FIG. 17B DDX3X and DDX3Y amino acid sequences aligned together. Nucleotide binding domains are indicated by rectangular boxes. Uniprot details for DDX3X (accession number: O00571): https://www.uniprot.org/uniprotkb/O00571/entry #sequences, and Uniprot details for DDX3Y (accession number: O15523): https://www.uniprot.org/uniprotkb/O15523/entry #sequences.

FIG. 18A-18C shows comparative helicase activities of DDX3X and DDX3Y using the ICD-helicase system. FIG. 18A Representative immunoblots of sgCTL and sgDDX3X 293T cells transfected with Flag-DDX3X/Y constructs, probed for Flag (for DDX3X/Y) and GAPDH (loading control). FIG. 18B Firefly luciferase activities of Rac1-FLuc reporter cells under indicated treatment conditions were determined by normalizing AcGFP⁺ levels within each sample before comparing against sgCTL firefly luciferase signals. FIG. 18C Firefly luciferase activities of DvL2-FLuc reporter cells under indicated treatment conditions were determined by normalizing AcGFP⁺ levels within each sample before comparing against sgCTL firefly luciferase signals. Cells were treated with cycloheximide as a translation inhibitory control. Data represent three independent experiments and values in the bars are mean±SEM. *, p<0.05; **, p<0.01; ****, p<0.0001; ns, non-significant.

FIG. 19A shows sgCTL and sgDDX3X 293T cells transfected with WT DDX3X or DDX3Y plasmids, as indicated, that were subjected to puromycin incorporation assay. Cells were lysed and cellular lysates were analysed for Flag expression by Western immunoblotting. The blot was also probed for GAPDH and stained with Ponceau S to confirm equal protein loading. FIG. 19B shows densitometric quantification of puromycin incorporation to determine relative levels of global protein synthesis. Data represent three independent experiments and values in the bars are mean±SEM. *, p<0.05; ****, p<0.0001.

FIG. 20A-20B shows representative immunoblots of sgDDX3X cells transfected with HA-tagged human DDX3X or mouse mDdx3x or mDdx3y and their relative luciferase luminescence activities: FIG. 20A Representative immunoblots of sgDDX3X cells transfected with vector alone or HA-tagged DDX3X (human), mDdx3x (mouse) or mDdx3y (mouse) plasmids. Cells were lysed and cellular lysates were analysed to determine the levels of HA (for DDX3) and GAPDH (loading control); FIG. 20B Firefly luciferase activity of sgCTL and sgDDX3X 293T cells expressing human DDX3X or mouse mDdx3, normalized using AcGFP⁺ levels within each sample before comparing against sgCTL firefly luciferase activity. Cells were treated with cycloheximide as an inhibitory control. Data represent three independent experiments and values are mean±SEM. ****, p<0.0001.

FIG. 21A-21D shows that both helicase and ATPase activities are necessary for DDX3Y function. Relative luciferase signals produced by WT or mutant DDX3Y as indicated in sgCTL and sgDDX3X Rac1-FLuc FIG. 21A and DvL2-FLuc FIG. 21B reporter cells, determined by normalizing AcGFP⁺ levels within each sample before comparing against control sgCTL firefly luciferase signals. Cells were treated with cycloheximide as an inhibitory control. FIG. 21C sgCTL and sgDDX3X 293T cells transfected with WT or mutant DDX3Y plasmids as indicated were subjected to puromycin incorporation assay, lysed and cellular lysates were analysed by Western immunoblotting. The blot was also probed for GAPDH and stained with Ponceau S to confirm equal protein loading. FIG. 21D Densitometric analysis of puromycin incorporation to determine relative levels of global protein synthesis. Data represent three independent experiments and values in the bars are mean±SEM. **, p<0.01; ****, p<0.0001; ns, non-significant.

FIG. 22A representative immunoblots of protein samples obtained from sgDDX3X cells transfected with empty vector or plasmids for Myc-elF4A1, Myc-elF4A2, HA-DDX5, Flag-DDX21, Flag-DHX36, or Myc-DDX17 and probed for Myc, HA, or Flag. Blots were re-probed for GAPDH as a loading control. FIG. 22B Firefly luciferase activity of sgCTL and sgDDX3X Rac1-Fluc as well as DvL2-FLuc reporter 293T cells expressing elF4A1 or elF4A2. FIG. 22C Firefly luciferase activity of control (sgCTL) and sgDDX3X Rac1-FLuc as well as DvL2-FLuc reporter 293T cells expressing DDX5, DDX317, DDX21, or DHX36. Firefly luciferase activity was normalized using AcGFP⁺ levels within each sample before comparing against sgCTL firefly luciferase activity. Cycloheximide was used as a positive control for translational inhibition. Data represent mean±SEM of 3 independent experiments. *, p<0.05; **, p<0.01; ****, p<0.0001.

FIG. 23A representative immunoblot of RWPE-1 cells overexpressing DDX3Y compared to cells electroporated with vector alone. It was noted that DDX3Y overexpression reduced DDX3X expression. GAPDH was used as a loading control. FIG. 23B Firefly luciferase activity of RWPE-1 cells overexpressing DDX3Y that were electroporated with Rac1-FLuc or DvL2-FLuc reporter plasmids, normalized against AcGFP⁺ levels within each sample. Cells were treated with cycloheximide as an inhibitory control. Data represent at least three independent experiments and values in the graph are mean±SEM. ****, p<0.0001.

FIG. 24A representative immunoblots of LNCaP cells expressing doxycycline (Dox) inducible shDDX3Y constructs for DDX3Y knockdown. GAPDH was used as a loading control. FIG. 24B Helicase activity of shCTL and shDDX3Y LNCaP cells expressing Rac1-FLuc reporter plasmid. Firefly luciferase activity of reporter cells, normalized against AcGFP⁺ levels within each sample before comparing against sgCTL firefly luciferase activity. Cycloheximide was used as an inhibitory control. Data represent at least three independent experiments and values in the graph are mean±SEM. ***, p<0.001; ns, non-significant.

### DETAILED DESCRIPTION

The following detailed description refers to, by way of illustration, specific details and embodiments in which the invention may be practiced. These embodiments are described in sufficient detail to enable those skilled in the art to practice the invention. Other embodiments may be utilized and structural and logical changes may be made without departing from the scope of the invention. Embodiments described below in context of the reporter cells are analogously valid for the respective methods, systems, uses, and vice versa. The various embodiments are not necessarily mutually exclusive, as some embodiments can be combined with one or more other embodiments to form new embodiments.

Unless otherwise defined, all technical and scientific terms used herein have the same meaning as commonly understood by one of ordinary skill in the art. The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will prevail. “About”, as used herein in connection with numerical values refers to the referenced numerical value ±10% or ±5%.

DDX3X is an ATP-dependent RNA helicase involved in diverse cellular processes. Somatic mutations in the DDX3X gene exhibit oncogenic or tumour suppressive activities, depending on cancer types. However, helicase-modulating roles of specific DDX3X mutations remain elusive mainly due to lack of an appropriate cell culture model system. Genome sequencing has identified numerous mutations in the DEAD-box RNA helicases, DDX3X and DDX3Y, associated with cancer and other diseases, but monitoring of their functional consequences remains a challenge. Conventional helicase assays are laborious, often technically difficult, and are performed in cell-free systems that do not address biologically relevant questions. Accordingly, there is still a void when it comes to the provision of alternative helicase assays, for a number of biopharmaceutical and biomedical applications.

To circumvent the challenges of conventional assays, the present application provides a novel, general strategy for interrogating helicase activities of DDX3 by developing a novel engineered DDX3 (i.e. DDX3X and DDX3Y) reporter cell-based system comprising an engineered reporter cell. The main aim of the present invention is the provision of a reporter cell that enables the efficient interrogation of helicase biology of DDX3 helicase.

In particular, to evaluate the helicase activity of the cellular DDX3 or its homologues, paralogs, or mutant variants in question, a reporter plasmid was developed to measure the luciferase signal corresponding to the helicase activity. Using 8 different point mutations in the DDX3X, identified in relapsed/refractory patients with diffuse large B cell lymphoma (DLBCL), it was shown that these DDX3X mutants are defective in helicase activity. Moreover, DDX3X mutations induced by the CRISPR knock-in technique in DLBCL cells abolished helicase activity of cells, validating the reporter cell-based system. The utility of the reporter cell disclosed herein is also shown to be extended to interrogate the Y chromosome-linked male paralog DDX3Y. In male lymphoma patients, ectopic DDX3Y expression can compensate for DDX3X loss to restore global protein synthesis. In this regard, it was noted that helicase activity of DDX3Y is less efficient compared to DDX3X, revealing subtle differences between the two paralog proteins despite having a high level of homology. Thus, the developed reporter cells disclosed herein can be applied widely to evaluate the effects of DDX3X mutations in diseases as well as study the biology of DDX3X and DDX3Y as well as their homologues, paralogues or mutational variants.

The cell-based reporter system disclosed herein may be referred to as an In-Cell DDX3 helicase (ICD-helicase) assay, and relies on an engineered reporter cell that lacks endogenous expression of the DDX3 helicase, and comprises a reporter expression construct comprising a reporter gene regulated by a untranslated region (UTR). Upon expression of the reporter gene, a detectable signal (e.g. luminescence if the reporter gene is a luciferase gene) is emitted that correlates with, and is indicative of, the helicase activity of the DDX3 helicase to be assessed.

Functionally active DDX3X and DDX3Y helicases regulate mRNA translation (and expression) of the reporter gene by unwinding a complex 5′ untranslated region (5′-UTR) secondary structure. Accordingly, the level of the emitted signal is proportional to the expression level of the reporter gene and correlates to helicase activity of the exogenously expressed DDX3X or DDX3Y or their homologues and variants thereof. Thus, in the reporter cells disclosed herein the detection of an emitted signal, and level, of the reporter gene is indicative that the exogenously expressed DDX3X has functional helicase activity by unwinding the 5′-UTR leading to the expression of the reporter gene.

The engineered reporter cell disclosed herein and assays applying the same, in contrast to the conventional approach of performing helicase unwinding assay or ATPase activity assay, offers several advantages. First, the ICD-helicase reporter cell system enables a quick and accurate screening of multiple pathogenic DDX3X and DDX3Y mutants. Second, the cell-based reporter system disclosed herein does not require recombinant proteins, saving time and resources in obtaining purified proteins. Finally, the reporter system disclosed herein is cell-based and provides a more accurate representation for cellular helicase unwinding activity of DDX3X and DDX3Y. Collectively, the disclosure herein highlight advantages of the developed ICD-helicase reporter cell system in studying the biology of DDX3 in health and DDX3 mutation-associated diseases.

Accordingly, there is provided herein an engineered reporter cell for use in assessing the activity of a DDX3 helicase, and methods of using the reporter cell that involves the interrogation of the activity of a DDX3 helicase.

As used herein, the term “DDX3 helicase” refers to a member of the DEAD-box protein family, characterized by the conserved amino acid motif D-E-A-D (Asp-Glu-Ala-Asp). DDX3 helicases are ATP-dependent RNA helicases involved in a variety of cellular processes, including RNA metabolism, transcription, translation, RNA splicing, and the regulation of RNA secondary structure. DDX3 helicase encompasses naturally occurring variants, homologs, paralogs and orthologs of DDX3 helicases across species, as well as engineered, mutational or synthetic versions of DDX3 helicase proteins. Variants may include proteins with minor sequence variations, such as polymorphisms, insertions, deletions, or substitutions that retain helicase activity or function in relevant biological processes. Homologs refer to DDX3-related helicases that share evolutionary ancestry and sequence homology, while paralogs are genes or proteins related by duplication within the same species that maintain helicase function and activity.

In various embodiments, the DDX3 helicase may be a human DDX3 family member including DDX3X and DDX3Y; a mouse DDX3 family member including Ddx3x and Ddx3y; a yeast helicase including Ded1 and Dbp1; a fruit fly helicase including Belle (Bel); a nematode helicase such as Ce-dbx-1; a plant DDX3 ortholog including AtRH30 and OsRH33; a viral DDX3 homolog; a DDX3 paralogue such as DDX4, DDX5, DDX17; and a variant, mutant and engineered form of these DDX3 helicases.

In various embodiments, the DDX3 helicase may be selected from DDX3X, DDX3Y, Ddx3x, Ddx3y, and their variants, homologues, and paralogues thereof. In various embodiments, the DDX3 helicase may be DDX3X, DDX3Y, and variants thereof.

DD3X helicase polypeptides and nucleic acids encoding DD3X helicase are readily known and available to the skilled person from publicly available sequence databases and include, those listed in the below Table 1 referencing relevant information. The indicated accession numbers can be used to retrieve specific sequences from NCBI and UniProt databases for cloning, experimental design, or mutagenesis to create mutational variants of the DDX3 helicase to be assessed by the present invention.

TABLE 1
Examples of DDX3 helicase sequences

| | Ref Amino acid | |
|---|---|---|
| Description | sequence | Ref Nucleotide sequence |
| Human DDX3X | UniProt: O00571 | NCBI Reference Sequence: |
| | | NG_012830.2 |
| | | Gene ID: 1654 |
| Human DDX3Y | UniProt: O15523 | GenBank: AF000984.1 |
| | | Gene ID: 8653 |
| Mouse Ddx3x | UniProt: Q62167 | GenBank: Z38117.1 |
| | | Gene ID: 13205 |
| Mouse Ddx3y | UniProt: Q62095 | GenBank: AJ007376.1 |
| | | Gene ID: 26900 |
| Yeast Ded1 | UniProt: P06634 | GenBank: X57278.1 |
| Drosophila Belle | UniProt: Q9V3C4 | GenBank: AF160911.1 |
| (Bel) | | |

The term “nucleic acid molecule” or “nucleic acid” or “nucleotide sequence” as used herein refers to a polymeric form of nucleotides of any length, either deoxyribonucleotides or ribonucleotides, or analogs thereof. Nucleic acid molecules may have any three-dimensional structure, and may perform any function, known or unknown. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. These can be DNA molecules or RNA molecules. They can exist as an individual strand, as an individual strand complementary to said individual strand, or as a double strand. With DNA molecules in particular, the sequences of both complementary strands in all three possible reading frames are to be considered in each case. Also to be considered is the fact that different codons, i.e. base triplets, can code for the same amino acids, so that a specific amino acid sequence (e.g. DDX3 helicase amino acid sequence) can be coded by multiple different nucleotide sequences. As a result of this degeneracy of the genetic code, all nucleic acid sequences that can encode the above-described amino acid sequences of DDX3 helicases are included in this subject of the invention. The skilled artisan is capable of unequivocally determining these nucleotide sequences, since despite the degeneracy of the genetic code, defined amino acids are to be associated with individual codons. The skilled artisan can therefore, proceeding from an amino acid sequence, readily ascertain nucleic acids coding for that amino acid sequence. In addition, in the context of nucleotide sequences according to the present invention one or more codons can be replaced by synonymous codons. This aspect refers in particular to expression of the DDX3 helicase described herein. For example, every organism, e.g. a host cell of a production strain, possesses a specific codon usage. “Codon usage” is understood as the translation of the genetic code into amino acids by the respective organism. Bottlenecks in protein biosynthesis can occur if the codons located on the nucleic acid are confronted, in the organism, with a comparatively small number of loaded tRNA molecules. Also it codes for the same amino acid, the result is that a codon becomes translated in the organism less efficiently than a synonymous codon that codes for the same amino acid.

By way of methods commonly known today such as, for example, chemical synthesis or the polymerase chain reaction (PCR) in combination with standard methods of molecular biology or protein chemistry, a skilled artisan has the ability to manufacture, on the basis of known DNA sequences and/or amino acid sequences, the corresponding nucleotide sequences all the way to complete genes. Such methods are known, for example, from Sambrook, J., Fritsch, E. F., and Maniatis, T, 2001, Molecular cloning: a laboratory manual, 3rd edition, Cold Spring Laboratory Press.

In various embodiments, the DDX3 helicase may be a “wild-type” or “naturally occurring” DDX3 helicase, and has the meaning commonly understood by those skilled in the art, which means a typical form of the DDX3 helicase that distinguishes it from mutants, derivatives or variant forms when it exists in nature, it can be isolated from natural sources and has not been deliberately modified. In various embodiments, the DDX3 helicase may be a variant, more particularly a mutational variant. The term “variant” in the context of the DDX3 helicase as used herein may refer to a DDX3 polypeptide comprising a modification or alteration and includes mutational variants. The modification or alteration may be a substitution, insertion, and/or deletion, at one or more (e.g., one or several) positions compared to the reference amino acid sequence. A substitution means replacement of the amino acid occupying a position with a different amino acid; a deletion means removal of the amino acid occupying a position; and an insertion means adding an amino acid adjacent to and immediately following the amino acid occupying a position. In this regard, variants of the amino acid sequence of DDX3 helicase herein may comprise a substitution, deletion, and/or insertion at one or more amino acid positions compared to the reference amino acid sequence. The amino acid changes may be of a minor nature, that is conservative amino acid substitutions or insertions that do not significantly affect the helicase activity of the protein. Such polypeptide variants are, for example, further developed by targeted genetic modification, i.e. by way of mutagenesis methods, and optimized for specific purposes or with regard to special properties. It is to be understood that the various polypeptide variants having at least one of the aforementioned deletions and/or mutations, even if their amino acid sequences are not explicitly described herein for the sake of conciseness, are contemplated to be within the scope of the present invention. The DDX3 helicase “variant” may be termed as a “functional variant” which refers to variants that retain full functionality of the original reference sequences. Said functional variants retain the functionality of the reference sequence and, in various embodiments, have at least the same or even higher activity than the sequence they are derived from. Generally, the term “variant” covers such DDX3 helicases that have at least 80%, or at least 90% sequence identity with the reference sequence over their entire length, preferably at least 80, 81, 82, 83, 84, 85, 86, 87, 88, 89, 90, 91, 92, 93, 94, 95, 96, 97, 98, or 99% sequence identity. The identity of nucleic acid sequences or amino acid sequences is generally determined by means of a sequence comparison. This sequence comparison is based on the BLAST algorithm that is established in the existing art and commonly used (cf. e.g. Altschul et al. (1990) “Basic local alignment search tool”, J. Mol. Biol. 215:403-410, and Altschul et al. (1997): “Gapped BLAST and PSI-BLAST: a new generation of protein database search programs”; Nucleic Acids Res., 25, p. 3389-3402) and is effected in principle by mutually associating similar successions of nucleotides or amino acids in the nucleic acid sequences and amino acid sequences, respectively. A tabular association of the relevant positions is referred to as an “alignment.” Sequence comparisons (alignments), in particular multiple sequence comparisons, are commonly prepared using computer programs which are available and known to those skilled in the art. Such an alignment is based on aligning similar nucleotide or amino acid sequences stretches with each other. Another algorithm known in the art for said purpose is the FASTA algorithm. Alignments, in particular multiple sequence comparisons, are typically done by using computer programs. Commonly used are the Clustal series (See, e.g., Chenna et al. (2003): Multiple sequence alignment with the Clustal series of programs. Nucleic Acid Research 31, 3497-3500), T-Coffee (See, e.g., Notredame et al. (2000): T-Coffee: A novel method for multiple sequence alignments. J. Mol. Biol. 302, 205-217) or programs based on these known programs or algorithms. Also possible are sequence alignments using the computer program Vector NTI® Suite 10.3 (Invitrogen Corporation, 1600 Faraday Avenue, Carlsbad, CA, USA) with the set standard parameters, with the AlignX module for sequence comparisons being based on the ClustalW. If not indicated otherwise, sequence identity relates to the entire length of the aligned sequence.

In various embodiments, the DDX3 helicase may be a mutational variant including but not limited to DDX3X mutant R475C, DDX3X mutant Y200H, DDX3X mutant R296C, DDX3X mutant Q309R, DDX3X mutant F357V, DDX3X mutant S470N, DDX3X mutant R534H, DDX3X mutant P568L, or DDX3X mutant comprising one or more of these amino acid substitutions.

### DDX3 Helicase Reporter Cells

In various embodiments, there is provided an engineered reporter cell for assessing the activity of DDX3 helicase, comprising a reporter expression construct that emits a detectable signal, that may be quantified, upon exogenous expression in the reporter cell of a functionally active DDX3 helicase. In various embodiments, the reporter expression construct may be comprised in a circular DNA molecule (i.e. a first circular DNA molecule) that has been introduced into a host cell.

As used herein, the term “assessing” may refer to the process of interrogating, measuring, evaluating, detecting, determining, or monitoring the activity, functionality, and expression, of the DDX3 helicase, that may yield qualitative and/or quantitative insights into the DDX3 helicase's role in cellular processes and disease mechanisms.

As used herein, the term “functionally active” of a DDX3 helicase refers to a DDX3 helicase that maintains and possesses the ability to unwind RNA or RNA/DNA hybrid secondary structures in an ATP-dependent manner, and in the context of the reporter cell disclosed herein will correlate with the presence, and quantifiable level, of an emitted detectable signal from the reporter gene product. As will be appreciated, if the DDX3 helicases expressed in the reporter cell disclosed herein, and the activity to be assessed, is not functionally active then this will be correlated with an absence, or low quantifiable level, of the emitted detectable signal.

In various embodiments, the reporter cell lacks endogenous expression of DDX3 helicase since this may interfere with assessment of the activity of the desired DDX3 helicase. In various embodiments, the reporter cell disclosed herein naturally or artificially lacks endogenous expression of the DDX3 helicase, more preferably the reporter cell is a DDX3 knock-out reporter cell. For example, in the event the DDX3 helicase is DDX3Y, then a cell being a female-derived line naturally lacks expression of the DDX3Y helicase (e.g. 293T cells). In various embodiments, endogenous DDX3 helicase is completely deleted or silenced in the reporter cell using a CRISPR/Cas9 system to provide a DDX3 knock-out reporter cell that lacks endogenous expression of said DDX3 helicase. A plasmid comprising and expressing one or more sgRNA sequences complementary to the target region in the gene to be knocked out (i.e. DDX3) may be introduced into the reporter cell to direct the Cas9 nuclease (encoded by and expressed from the plasmid) to the specific location in the DNA through complementary base pairing. Thus, the sgRNAs serve as the targeting component of the CRISPR-Cas9 system, guiding the Cas9 nuclease to specific genomic loci for gene knockout. For example, to knock-out human DDX3X a first sgRNA may target a genomic nucleotide sequence set forth in SEQ ID NO:20 and a second sgRNA may target a genomic nucleotide sequence set forth in SEQ ID NO: 21.

In various embodiments, the DDX3 helicase to be assessed may be exogenously or transiently expressed in the reporter cell, and the activity of the DDX3 helicase may be unknown thus providing the need for such assessment of helicase activity. Alternatively, the activity of the DDX3 helicase to be assessed in the reporter cell may be known (i.e. the DDX3 helicase may be known to be functionally active), whereby the impact of other cellular components/mechanism and/or agents administered to the reporter cell on the DDX3 helicase activity may be interrogated, such as possible candidate agents that may inhibit or stimulate or have limited effect on the helicase activity.

In various embodiments, the DDX3 helicase is exogenously or transiently expressed in the reporter cell. In this regard, the exogenous or transient expression of the DDX3 helicase can be achieved through any method or technique known to those skilled in the art, and generally includes introducing a recombinant DNA construct encoding the DDX3 helicase gene into the reporter cell, allowing the reporter cell to exogenously or transiently express and produce the DDX3 helicase protein within the cell. In various embodiments, the DDX3 helicase is exogenously or transiently expressed in the reporter host cell through plasmid transfection, which may involve a nucleotide sequence encoding the DDX3 being inserted into an expression plasmid under the control of a promoter (e.g., CMV, EF-1α) that is compatible with the reporter cell. The plasmid may also contain tags for detection (e.g., His-tag, FLAG-tag) and antibiotic resistance for selection. As will be appreciated, the inclusion of a nucleotide sequence encoding a FLAG tag (SEQ ID NO: 1—DYKDDDDK) in the plasmid may be beneficial for detection, purification and analysis of the DDX3 helicase.

In various embodiments, a circular DNA molecule (i.e. a second circular DNA molecule) may be introduced into the reporter cell, and comprises a nucleotide sequence encoding the DDX3 helicase, and one or more regulatory elements capable of controlling transient or exogenous expression of the DDX3 helicase within the cell. In various embodiments, the expression of the DDX3 helicase may be induced or controlled such that the DDX3 helicase protein is produced at a desired time during said assessment. In various embodiments, the expression of the DDX3 may be controlled and driven by a cytomegalovirus (CMV) promoter and the circular DNA molecule may include a nucleotide sequence encoding a Flag tag at the N-terminus of the nucleotide sequence encoding the DDX3 helicase.

In various embodiments, the nucleotide sequence encodes a human DDX3 helicase (i.e. DDX3X or DDX3Y), or a mouse DDX3 helicase (i.e. Ddx3x and Ddx3y), or a variant of these DDX3 helicases. In various embodiments, the nucleotide sequence encodes a human DDX3X (SEQ ID NO: 2), a human DDX3Y (SEQ ID NO:3), a mouse Ddx3x (SEQ ID NO:4), a mouse Ddx3y (SEQ ID NO: 5), or variants thereof. It will be appreciated that the “variants” disclosed herein share a % sequence identity or % sequence homology with the reference amino acid sequence set forth in SEQ ID NO: 2-5.

In various embodiments, the expression of the DDX3 helicase, may be constitutive or stringently controlled by an inducible expression system such as the Tet on/off system that is well-known in the art. In various embodiments, the expression of the DDX3 helicase is constitutively expressed once the second circular DNA molecule (i.e. plasmid) is introduced into the reporter cell. In various embodiments, the DDX3 helicase is stably expressed in the reporter cell wherein the second circular DNA molecule is a stable extrachromosomal element.

In various embodiments, the one or more regulatory elements capable of controlling transient or exogenous expression of the DDX3 helicase within the cell may comprise a leader, polyadenylation sequence, promoter, enhancer or upstream activating sequence, and transcription terminator. In various embodiments, the promoter may be a constitutive promoter, or an inducible promoter. A “constitutive promoter” is a promoter that is active under most environmental and physiological conditions. An “inducible promoter” is a promoter that is active under specific environmental or physiological conditions.

In various embodiments, the circular DNA molecule is an expression plasmid for transient or exogenous expression of the DDX3 helicase. The preparation and provision of the plasmid that is capable of transiently or exogenously expressing the DDX3 helicase, as well as methods to introduce/transfect a host cell with the plasmids are well known in the art and can be routinely applied by those skilled in the art. In various embodiments, the circular DNA molecule may be an episomal (extrachromosomal) expression plasmid.

In various embodiments, there is provided herein a reporter cell for assessing the activity of DDX3 helicase, comprising a first circular DNA molecule comprising a reporter expression construct disclosed herein, wherein the reporter cell lacks endogenous expression of the DDX3 helicase, optionally the reporter cell comprises a second circular DNA molecule comprising a nucleotide sequence encoding the DDX3 helicase to be assessed, and one or more regulatory elements capable of controlling transient or exogenous expression of the DDX3 helicase within the cell.

The reporter cell may be developed and engineered from any suitable host cell. As used herein, the term “host cell” refers to a living cell into which the reporter expression construct is to be or has been introduced. The living cell includes both a cultured cell and a cell within a living organism. In various embodiments, host cells can be engineered to incorporate a desired gene (i.e. encoding DDX3 helicase) for exogenous expression and also engineered to delete or knock-out or knock-down endogenous expression of a desired gene ((i.e. encoding DDX3 helicase). The host cell disclosed herein may be any eukaryotic host cell. In various embodiments, the eukaryotic host cell is a higher eukaryotic host cell. The term “higher eukaryotic cell” as used herein refers to eukaryotic cells that are not cells from unicellular organisms. In other words, a higher eukaryotic cell is a cell from (or derived from, in case of cell cultures) a multicellular eukaryote such as a human cell, human cell line or another mammalian cell or cell line. Particularly, the term generally refers to mammalian cells, human cells and insect cells. More particularly, the term refers to vertebrate cells, even more particularly to mammalian cells or human cells.

In various embodiments, the eukaryotic host cell is a mammalian cell. The mammalian cell lines can include, but are not limited to a human, simian, murine, mice, rat, monkey, rabbit, rodent, hamster, goat, bovine, sheep or pig cell lines. In various embodiments, the host cell is a human cell.

In various embodiments, the host cell may be a cell derived from a subject or a cell line, preferably the cell is a primary cell derived from a human or a human cell derived from a cell line selected from Hela cells and human embryonic kidney (HEK-293) cells, diffuse large B-cell lymphoma cells (U2932), prostate cell line (RWPE-1, DU145, 22Rv1, LNCaP) and derivatives thereof. However, it will be appreciated that any other cultured cells or cell types can also be used for studying DDX3 activity depending on the specific research intention and investigation being carried out.

In various embodiments, the host cell may be a human cell or a human cell line, more preferably an embryonic kidney 293 (HEK 293) cell or derivatives, even more preferably a HEK293T cell.

In various embodiments, the host cell may be a primary cell derived from a subject with a disease or condition such as cancer, preferably the primary cell is obtained from a cancer sample. Accordingly, the reporter cell disclosed herein may be used to interrogate and assess the activity of DDX3 helicase in a cancer cell that may be useful in understanding aspects underlying cancer and the role of DDX3 helicase in the disease state and progression.

The term “subject”, as used herein may refer to a warm-blooded animal, preferably a mammal, more preferably a human. Said subject may be awaiting or receiving medical treatment for cancer, or is, or will become the subject of a medical procedure, or is being monitored for the development of cancer treatable with modulators of DDX3 helicase activity. Subjects include those already being afflicted by cancer as well as subjects susceptible to the progression of the cancer or for whom cancer or progression of cancer should be prevented or delayed. In various embodiments, the subject has been diagnosed with cancer, and the diagnosis was based on standard clinical endoscopic, radiological and histological criteria. In various embodiments, the subject may be healthy and has not been diagnosed with any disease or condition.

In various embodiments, the host cell may be derived from a subject, and more particularly a sample obtained from the subject. The term “sample,” as used herein, refers to a composition that is obtained or derived from the subject that contains a cellular and/or other molecular entity that is to be characterized and/or identified, for example, based on physical, biochemical, chemical, and/or physiological characteristics. For example, the phrase “disease sample” and variations thereof (i.e. cancer or tumour sample) refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumour lysates, and tissue culture medium, tissue extracts such as homogenized tissue, tumour tissue, cellular extracts, and combinations thereof.

In various embodiments, the cancer may be selected from adrenal cancer, acute lymphoblastic leukemia, acute myelogenous leukemia, astrocytoma, basal cell carcinoma, bile duct cancer, bladder cancer, bone cancer, breast cancer, brain cancer, carcinoma, cardiac tumor, cervical cancer, childhood cancers, chronic lymphocytic leukemia, chronic myelogenous leukemia, colorectal cancer, embryonal tumor, epithelial cancer, esophageal cancer, gastrointestinal cancer, germ cell tumor, gallbladder cancer, gastric cancer, glioma, head and neck cancer, haematological malignancy, Hodgkin's lymphoma, non-Hodgkin's lymphoma, intestinal cancer, intraocular melanoma, kidney cancer, laryngeal cancer, leukemia, lung cancer, liver cancer, malignant peripheral nerve sheath tumor, melanoma, mesothelioma, nasopharyngeal carcinoma, neuroblastoma, neurofibroma, oral cancer, non-small cell lung cancer, osteosarcoma, ovarian cancer, pituitary tumor, prostate cancer, pancreatic cancer, retinoblastoma, rhabdomyosarcoma, sarcoma, small cell lung cancer, testicular cancer, throat cancer, thyroid cancer, transitional cell carcinoma, urogenital cancer, urothelial carcinoma, uterine cancer, vaginal cancer, or Wilms' tumor.

In various embodiments, the host cell is a cancer cell, more preferably the cancer cell may be a lymphoma cell or cell line, more preferably a non-Hodgkin's lymphoma (non-Hodgkin lymphoma or NHL) cell or cell line.

In various embodiments, the reporter expression construct may be comprised in a circular DNA molecule.

In various embodiments, the circular DNA molecule may be a plasmid, vector, cosmid, bacterial artificial chromosome (BAC), bacteriophage, viral vector or hybrids thereof. In various embodiments, the circular DNA molecule is a vector, more particularly an expression vector.

As used herein, the term “vector” may be understood for purposes herein as elements-made up of nucleic acids-that contain the reporter expression construct contemplated herein as a characterizing nucleic acid region. They enable said nucleic acid to be established as a stable genetic element in a species or a cell line over multiple generations or cell divisions. In particular, when used in bacteria, vectors are special plasmids, i.e. circular genetic elements. Included among the vectors are, for example, those whose origins are bacterial plasmids, viruses, or bacteriophages, or predominantly synthetic vectors or plasmids having elements of widely differing derivations. Using the further genetic elements present in each case, vectors are capable of establishing themselves as stable units in the relevant host cells over multiple generations. They can be present extrachromosomally as separate units, or can be integrated into a chromosome.

Expression vectors encompass nucleic acid sequences which are capable of replicating in the host cells. In various embodiments, the vectors described herein thus also contain regulatory elements that control expression of the nucleic acids. Expression is influenced in particular by the promoter or promoters that regulate transcription. Expression can occur in principle by means of the natural promoter originally located in front of the nucleic acid to be expressed, but also by means of a host-cell promoter furnished on the expression vector or also by means of a modified, or entirely different, promoter of another organism or of another host cell. In the present case at least one promoter for expression of a nucleic acid as contemplated herein is made available and used for expression thereof. Expression vectors can furthermore be regulated, for example by way of a change in culture conditions or when the host cells containing them reach a specific cell density, or by the addition of specific substances, in particular activators of gene expression. The expression vector may be based on plasmids well known to person skilled in the art such as pBR322, puC 16, pBluescript (RTM) and the like. Thus, the expression vector may be termed as an expression plasmid. In various embodiments, the circular DNA molecule may be an expression plasmid for transient expression of the expression construct. The preparation and provision of the plasmid comprising the reporter expression construct disclosed herein, as well as methods to introduce/transfect a host cell with the plasmids are well known in the art and can be routinely applied by those skilled in the art.

In various embodiments, the circular DNA molecule comprising the reporter expression construct (i.e. a first circular DNA molecule) may be an expression plasmid. The preparation and provision of the plasmid that is capable of expressing the reporter expression construct and reporter gene (i.e. upon unwinding of the 5′-UTR), as well as methods to introduce/transfect a host cell with the plasmids, are well known in the art and can be routinely applied by those skilled in the art. In various embodiments, the circular DNA molecule comprising the reporter expression construct may be an episomal (extrachromosomal) expression plasmid.

In various embodiments, the circular DNA molecule comprising the reporter expression construct may be a viral vector, more particularly a lentiviral vector such as a pLVX vector or a pLJM1 vector.

In various embodiments, the reporter expression construct comprises:
i) a reporter gene operably linked to one or more regulatory elements capable of controlling and/or enhancing expression of the reporter gene; and
ii) a nucleotide sequence encoding a 5′ untranslated region (5′-UTR), wherein unwinding of a secondary structure of the 5′-UTR by a functionally active DDX3 helicase facilitates expression of the reporter gene and emission of a detectable signal by the reporter gene product, that may be qualitatively and/or quantitatively measured and correlated with the activity of the DDX3 helicase expressed in the cell.

The term “operably linked” as used herein refers to the relationship between two or more nucleotide sequences that interact physically or functionally. For example, a promoter or regulatory nucleotide sequence is said to be operably linked to a nucleotide sequence that codes for an RNA or a protein if the two sequences are situated such that the regulatory nucleotide sequence will affect the expression level of the coding or structural nucleotide sequence. “Regulatory nucleotide sequences” as used herein refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; and polyadenylation recognition sequences. Particular regulatory sequences may be located upstream and/or downstream of a coding sequence operably linked thereto.

In various embodiments, the “reporter gene” disclosed herein refers to a nucleotide sequence and genetic element that encodes a product/protein capable of producing and emitting a detectable signal upon expression. The signal emitted by the reporter gene product is easily measurable and may correlate directly with the activity of the DDX3 helicase. In particular, upon expression, the reporter gene product (e.g., luciferase enzyme) catalyses a biochemical reaction that produces a quantifiable signal, such as bioluminescence, fluorescence, or colorimetric change. A quantifiable, visible, or measurable output of the reporter gene expression correlates with the activity of the DDX3 helicase. The detectable signal emitted by the reporter gene product may be measured using appropriate well-known equipment (e.g., luminometer for luciferase), allowing qualitative/quantitative analysis.

In various embodiments, the reporter gene may be a luminescent reporter gene (i.e. luciferase gene) whereby the reporter gene product emits a detectable luminescent signal when expressed. Luciferase genes from a wide variety of vastly different species, particularly the luciferase genes of Photinus pyralis (the common firefly of North America), Pyrophorus plagiophthalamus (the Jamaican click beetle), Renilla reniformis (the sea pansy), and several bacteria (e.g., Xenorhabdus luminescens and Vibrio spp.), are extremely popular luminescence reporter genes. Luciferase genes are widely used as genetic reporters due to the non-radioactive nature, sensitivity, and extreme linear range of luminescence assays. For instance, as few as 10-20 moles of firefly luciferase can be detected.

In various embodiments, the reporter gene may be selected from a gene encoding firefly luciferase (FLuc), a gene encoding renilla luciferase (Rluc), a gene encoding Cypridina Luciferase (Cluc), a gene encoding Gaussia Luciferase (Gluc), or a gene encoding NanoLuc Luciferase (NanoLuc).

In various embodiments, the reporter gene may be a gene encoding firefly luciferase (FLuc). In various embodiments, the gene encoding firefly luciferase comprises or consists of a nucleotide sequence set forth in SEQ ID NO:7, or functional variants thereof. These Fluc functional variants in general include nucleotide sequences derived from the reference Fluc sequence suitable for realizing the intended use as a reporter gene.

In various embodiments, the 3′-end of the reporter expression construct may be polyadenylated with a polyA sequence. In various embodiments, the 5′ end of the reporter expression construct is not capped. In various embodiments, the 3′-end of the reporter expression construct further comprises a poly(A) sequence comprising 20 to about 400 adenosine nucleotides. In various embodiments, the poly(A) sequence is downstream of the reporter gene.

In various embodiments, the reporter gene may be operably linked to a promoter for controlling expression of the reporter gene. In various embodiments, the “promoter” is any promoter that is highly capable and efficient at initiating transcription and producing a large amount of RNA from the reporter gene and corresponding protein. Specifically, the term “promoter” refers to a region of DNA that may be upstream from the start of transcription, and that may be involved in recognition initiation of transcription. The promoter may be derived from the genomes of viruses, such as polyoma virus, adenovirus, fowlpox virus, bovine papilloma virus, avian sarcoma virus, cytomegalovirus (CMV), retrovirus and Simian Virus 40 (SV40), or from heterologous mammalian promoters. Many promoters known in the art can be used for expression in host cells.

In various embodiments, the reporter gene may be operably linked to a constitutive promoter. Many constitutive promoters are known in the art and can be used for expression in host cells of genes and particularly reporter genes. Examples of constitutive promoters include, but are not limited to, the promoter of the mouse metallothionein I gene sequence; the TK promoter of Herpes virus, the SV40 early promoter; the yeast gall gene sequence promoter, the CMV promoter, the EF-1 promoter, the actin promoter, the phosphoglycerate kinase promoter, the ubiquitin promoter and the thymidine kinase promoter, the ecdysone-responsive promoter(s), tetracycline-responsive promoter, and the like. In various embodiments, the constitutive promoter is a CMV promoter, SV40 promoter, EF-1 promoter, or an actin promoter. In various embodiments, the constitutive promoter may be selected from a CMV promoter, SV40 promoter or a TK promoter, preferably the constitutive promoter is a cytomegalovirus (CMV) promoter. In various embodiments, the constitutive promoter is a CMV promoter comprising or consisting of a nucleotide sequence set forth in SEQ ID NO: 6, or functional variants thereof. The functional variant in general includes nucleotide sequences derived from the reference promoter sequence suitable for realizing the intended use as a constitutive promoter.

In various embodiments, the reporter gene may be positioned downstream of the constitutive promoter.

The term “downstream” as used herein refers to a nucleotide sequence that is located 3′ to a reference nucleotide sequence. In particular, downstream nucleotide sequences generally relate to sequences that follow the starting point of transcription. For example, the translation initiation codon of a gene is located downstream of the start site of transcription. The term “immediately downstream” may be used to specify that the nucleotide sequence immediately follows and is directly next to the reference nucleotide sequence in the 3′ direction, with no other intervening nucleotide sequence or genetic element therein between. The term “upstream” as used herein refers to a nucleotide sequence that is located 5′ to a reference nucleotide sequence. In particular, upstream nucleotide sequences generally relate to sequences that are located on the 5′ side of a coding sequence or starting point of transcription. For example, most promoters are located upstream of the start site of transcription. The term “immediately upstream” may be used to specify that the nucleotide sequence immediately precedes and is directly next to the reference nucleotide sequence in the 5′ direction, with no other intervening nucleotide sequence or genetic element therein between.

In various embodiments, the elements in the expression construct (i.e. reporter gene. 5-UTR sequence, IRES, GFP) may be separated by an intervening nucleotide sequence that may be termed as a spacer sequence. The spacer sequence may be termed as 5′ spacer sequence or a 3′ spacer sequence dependent on the positional relationship to an element in the expression construct. For example, the reporter expression construct may comprise a spacer sequence between the nucleotide sequence encoding the 5′-UTR and the reporter gene (i.e. spacer sequence that links the 3′ end of the 5′ UTR sequence and the 5′ end of the reporter gene). For example, multiple cloning sites (MOS) or restriction sites may be added to allow insertion of other S-UTRs or genes for additional functionalities. The spacer sequence may be a short spacer sequence that is less than or equal to 30 nucleotides (≤30 nt) in length, or a long spacer sequence that is more than 30 nucleotides (>30 nt) in length. In various embodiments, the spacer sequence is a short spacer sequence of at least 2, 3, 4, 5, 6, 7, 8, 9, 10, 11, 12, 13, 14, 15, 16, 17, 18, 19, 20, 25 or 30 nucleotides in length.

In various embodiments, the 5′-UTR nucleotide sequence may be positioned upstream of the reporter gene (i.e., CMV-5′-UTR-FLuc). In various embodiments, the reporter expression construct does not comprise a nucleotide sequence encoding a 3′-UTR.

In various embodiments, the reporter expression construct may comprise from 5′ to 3′ direction: the constitutive promoter, the 5′-UTR, and the reporter gene. In various embodiments, the reporter expression construct comprises a short spacer sequence between the 5′-UTR and the reporter gene.

As used herein, the term “UTR” refers to the untranslated region of a mRNA, i.e., the region of the mRNA that is not translated into protein. A 5′ UTR refers to a region upstream of a coding region that encodes a stable RNA or a functional protein. A 5′-UTR secondary structure refers to the three-dimensional arrangement of the nucleotide sequence within the 5′-UTR, resulting from the formation of base pairs between complementary nucleotides within the sequence. This secondary structure refers to RNA duplexes that form stems, loops, hairpins, bulges, and other complex shapes. In various embodiments, the 5′ UTR may comprise a stable hairpin secondary structure In this regard, the 5′-UTR is the region between the transcription start site (usually at position 1) and the start codon (AUG) of the gene. Accordingly, the 5′ UTR may be derived from any suitable gene so long as the 5′-UTR and its secondary structure is known to be regulated by DDX3 helicase, whereby said DDX3 helicase is capable of unwinding the secondary structure of the 5′-UTR.

Once the nucleotide sequence encoding the 5′-UTR is transcribed into mRNA, it can form secondary structures that are capable of being unwound by functionally active DDX3 helicases. The 5′-UTR nucleotide sequences in the reporter expression construct are transcribed into RNA during gene expression, and these RNA UTRs fold into secondary structures that regulate the post-transcriptional process that leads to the translation and production of the reporter gene product and emitted detectable signal. As such, the 5′-UTR may be termed as a DDX3 helicase-sensitive 5′ UTR, whose secondary structure is capable of being regulated (i.e. unwound) by the DDX3 helicase and thus measure DDX3 helicase-mediated translational activity in the reporter cell.

In various embodiments, the secondary structure of the 5′-UTRs refers to an RNA duplex including a combination of stem-loops, bulges, internal loops, and possibly G-quadruplexes.

In various embodiments, the nucleotide sequence encoding the 5′-UTR may be derived from a gene selected from ATF5, RPLP1, DVL2, PRKRA, RAC1, ODC1 and CCNE1. In various embodiments, the nucleotide sequence encoding the 5′-UTR may comprise or consist of a nucleotide sequence as set forth in any one of SEQ ID NO: 12-19 or functional variants thereof.

In various embodiments, the nucleotide sequence encoding the 5′-UTR may be derived from a 5′-UTR of the gene RAC1, DVL2 or variants thereof. In various embodiments, the 5′-UTR may comprise or consist of a nucleotide sequence as set forth in any one of SEQ ID NO: 14, 15 and 17 or functional variants thereof. The term “functional variant” of these 5′-UTR sequences as used herein relates to a nucleotide sequence encoding the 5′-UTR derived from a gene selected from ATF5, RPLP1, DVL2, PRKRA, RAC1, ODC1 and CCNE1 having one or more substitutions, deletions or additions, preferably two, three, four, five or six in contrast to the reference nucleotide sequences. The term “functional variant” also relates to any fragments of the reference nucleotide sequences. The term “functional variant” in general includes nucleotide sequences derived from the reference sequences suitable for realizing the intended use of the present invention, which means that the functional variant sequences encode a 5′-UTR with a secondary structure that is capable of being regulated by DDX3 helicase, whereby said DDX3 helicase is capable of unwinding the secondary structure of the 5′-UTR

In various embodiments, the reporter expression construct further comprises one or more of the following elements: an iron response element (“IRE”), Internal ribosome entry site (“IRES”), upstream open reading frame (“uORF”), male specific lethal element (“MSL-2”), G quartet element, 5′-terminal oligopyrimidine tract (“TOP”), AU-rich element (“ARE”), selenocysteine insertion sequence (“SECIS”), histone stem loop, cytoplasmic polyadenylation element (“CPE”), nanos translational control element, amyloid precursor protein element (“APP”), translational regulation element (“TGE”)/direct repeat element (“DRE”), Bruno element (“BRE”), and a 15-lipoxygenase differentiation control element (“15-LOX-DICE”).

In various embodiments, the reporter expression construct further comprises an Internal Ribosome Entry Site (IRES). In various embodiments, the IRES may be positioned downstream of the reporter gene. The internal ribosome entry site (“IRES”) is one of the 5′ UTR-based cis-acting elements of post-transcriptional gene expression control. IRESes facilitate cap-independent translation initiation by recruiting ribosomes directly to the mRNA start codon. IRESes are commonly located in the 3′ region of a 5′ UTR and are frequently composed of several discrete sequences. IRESes do not share significant primary structure homology, but do form distinct RNA secondary and tertiary structures. Some IRESes contain sequences complementary to 18S RNA and therefore may form stable complexes with the 40S ribosomal subunit and initiate assembly of translationally competent complexes. A classic example of an “RNA-only” IRES is the internal ribosome entry site from Hepatitis C virus. However, most known IRESes require protein co-factors for activity. More than 10 IRES trans-acting factors (“ITAFs”) have been identified so far. In addition, all canonical translation initiation factors, with the sole exception of 5′ end cap-binding elF4E, have been shown to participate in IRES-mediated translation initiation (reviewed in Vagner et al., 2001, EMBO reports 2:893 and Translational Control of Gene Expression, Sonenberg, Hershey, and Mathews, eds., 2000, CSHL Press). In various embodiments, the IRES comprises or consists of a nucleotide sequence as set forth in SEQ ID NO:8, or functional variants thereof. These IRES functional variants in general include nucleotide sequences derived from the reference IRES sequence suitable for realizing the intended use as an IRES.

In various embodiments, the reporter expression construct further comprises a fluorescent marker gene that may function as an internal control to normalize possible variations in transfection efficiency. The term “fluorescent marker gene” disclosed herein refers to a gene or nucleotide sequence whose expression in a host cell can be detected or made visible. In various embodiments, the fluorescent marker gene is selected from green fluorescent protein (GFP) or enhanced green fluorescent protein (eGFP), yellow fluorescent protein (YFP) or enhanced yellow fluorescent protein (eYFP), red fluorescent protein (RFP), mCherry, mRaspberry, mPlum, mTomato, or dsRed. In various embodiments, the fluorescent marker gene is a green fluorescent protein, more preferably a Aequorea cerulescens green fluorescent protein (AcGFP). In various embodiments, the fluorescent marker gene is AcGFP gene comprising or consisting of a nucleotide sequence set forth in SEQ ID NO: 9, or functional variants thereof. These AcGFP functional variants in general include nucleotide sequences derived from the reference AcGFP sequence suitable for realizing the intended use as a fluorescent reporter gene.

In various embodiments, the fluorescent marker gene may be positioned downstream of the reporter gene.

In various embodiments, the reporter expression construct may comprise:
i) a reporter gene operably linked to a constitutive promoter capable of controlling the expression of the reporter gene;
ii) a nucleotide sequence encoding a 5′ untranslated region (5′-UTR) positioned upstream of the reporter gene such that unwinding of a secondary structure of the 5′-UTR by a functionally active DDX3 helicase facilitates the expression of the reporter gene and emission of a detectable signal;
iii) an Internal Ribosome Entry Site (IRES); and
iv) a fluorescent marker gene,
wherein the IRES is positioned downstream of the reporter gene, and the fluorescent marker gene is positioned downstream of the IRES (i.e. the IRES is positioned between the reporter gene and the fluorescent marker gene).

In various embodiments, the reporter expression construct may comprise from 5′ to 3′ direction: the 5′-UTR nucleotide sequence, and the reporter gene, the IRES, and the fluorescent marker gene.

In various embodiments, the reporter expression construct may comprise an antibiotic-resistance gene for selection of reporter cells. This allows only cells successfully introducing the circular DNA molecule (and reporter expression construct) to survive and get selected for use as a reporter cell disclosed herein. In various embodiments, the antibiotic resistance gene may be a gene that confers resistance to neomycin (G418), hygromycin, blasticidin, puromycin, bleomycin and phleomycin. In various embodiments, the antibiotic resistance gene may be a blasticidin resistance gene that comprises or consists of a nucleotide sequence as set forth in SEQ ID NO:10, or functional variants thereof. These antibiotic-resistance gene variants in general include nucleotide sequences derived from the reference sequence suitable for realizing the intended use as an antibiotic-resistance gene. In various embodiments, the antibiotic-resistance gene is positioned downstream of the reporter gene, more particularly downstream of the fluorescent marker gene. In various embodiments, the antibiotic-resistance gene may be under control by a PGK or other relevant promoter, such as CMV, HTLV, TRE-tight, or EF1α/HTLV composite promoter.

In various embodiments, the reporter expression construct may comprise from 5′ to 3′ direction: the 5′-UTR nucleotide sequence, the reporter gene, the IRES, the fluorescent marker gene, and the antibiotic-resistance gene.

As will be appreciated, the reporter cell disclosed herein may be expanded in a cell culture medium suitable for expansion of the reporter cell. Accordingly, there is also provided a reporter cell culture comprising a plurality of the reporter cells disclosed herein.

### Method of Producing the DDX3 Helicase Reporter Cell

There is also provided a method of producing the reporter cell disclosed herein comprising:
a) providing a host cell that lacks endogenous expression of the DDX3 helicase; and
b) introducing a circular DNA molecule into the host cell at suitable conditions to induce uptake of the circular DNA molecule into the cell, wherein the circular DNA molecule comprises a reporter expression construct disclosed herein.

All embodiments disclosed above in relation to the reporter cell similarly apply to the method of production and vice versa.

In various embodiments, the step of introducing the circular DNA molecule into the host cell, may be via any means available in the art, including but not limited to DNA transfection, transduction, transformation, viral transduction, biolistic technology, ultrasound, nanoparticles, microinjection, or HIV Tat-mediated polypeptide delivery.

In various embodiments, the step of introducing the circular DNA molecule into the host cell may comprise transfecting the circular DNA molecule into the host cell at suitable conditions to induce said transfection.

The term “transfection” as used herein means the uptake of exogenous or heterologous RNA or DNA by a cell. A cell has been “transfected” by exogenous or heterologous RNA or DNA when such RNA or DNA has been introduced inside the cell. Methods to co-transfect eukaryotic host cells are well known in the art and can be routinely applied by those skilled in the art. Transfection conditions, reagents and mediums can be selected by those skilled in the art based on the host organism used by resorting to general knowledge and techniques known in the art.

In various embodiments, the method may further comprise introducing a second circular DNA molecule into the cell at suitable conditions to induce uptake of the second circular DNA molecule into the cell, wherein the second circular DNA molecule comprises a nucleotide sequence encoding the DDX3 helicase, and one or more regulatory elements capable of controlling and/or enhancing transient or exogenous expression of the DDX3 helicase. In various embodiments, the second circular DNA molecule is introduced into the host cell and may contain inducible functionalities for stable expression of the DDX3 helicase, wherein the second circular DNA molecule is a stable extrachromosomal element with expression of the DDX3 helicase being inducible prior to or during assaying for the DDX3 helicase activity. However, it will be appreciated that the second circular DNA molecule may be introduced into the produced reporter cell prior to assaying for the DDX3 helicase activity.

In various embodiments, introducing the second circular DNA molecule into the host cell may comprise transfecting the second circular DNA molecule into the host cell at suitable conditions to induce said transfection. In various embodiments, the second circular DNA molecule may be introduced into the host cell only after the first circular DNA molecule has been introduced into the host cell.

In various embodiments, if the host cell does not naturally lack endogenous expression of the DDX3 helicase, prior to the providing step the method may comprise artificially engineering the host cell to lack endogenous expression of the DDX3 helicase, which may include the use of one or more well-known techniques such as Gene Knock-Out via CRISPR-Cas9, RNA Interference (RNAi), CRISPR Interference (CRISPRi), Gene Knock-Out via Homologous Recombination, or Antisense Oligonucleotides (ASOs). As a result the reporter cell completely (i.e. Knock-Out) or nearly completely (i.e. Knock-down) lacks endogenous expression of the DDX3 helicase.

In various embodiments, the host cell may be a DDX3 knock-out cell. The term “knock-out cell” or KO cell, as used herein refers to a cell in which the endogenous expression of a specific gene (i.e. DDX3 helicase) has been completely disrupted or eliminated. This typically involves mutating, deleting, or otherwise disabling the gene at the DNA level, preventing it from being expressed and thus producing its corresponding protein. Producing such a KO cell may be carried out using any well-known method or technique to those skilled in the art. In various embodiments, endogenous DDX3 helicase may be completely disrupted or eliminated in the cell using a CRISPR/Cas9 system to provide a DDX3 knock-out cell that lacks endogenous expression of said DDX3 helicase.

In various embodiments, the lack of endogenous expression of the DDX3 helicase may be analysed and confirmed by protein expression analysis (i.e. Western Immunoblotting, ELISA), mRNA expression analysis (i.e. qRT-PCR, RNA-seq), and/or genotypic confirmation (i.e. PCR and sequencing analysis).

In various embodiments, the method of producing the reporter cell disclosed herein may comprise:
providing a eukaryotic host cell that lacks endogenous expression of the DDX3 helicase;
transfecting a first circular DNA molecule into the eukaryotic host cell at suitable conditions to induce said transfection, wherein the circular DNA molecule comprises a reporter expression construct disclosed herein; and
transfecting a second circular DNA molecule into the eukaryotic host cell at suitable conditions to induce said transfection, wherein the second circular DNA molecule comprises a nucleotide sequence encoding the DDX3 helicase, and one or more regulatory elements capable of controlling and/or enhancing transient or exogenous expression of the DDX3 helicase.

In various embodiments, the transfection of the above-mentioned circular DNA molecules in the host cell may be carried out simultaneously (co-transfection) or sequentially. The transfection of the first circular DNA molecule may be carried out first, in order to obtain stable transfectants, that are later used for a second transfection of the second circular DNA molecule to obtain double transfectants (transient or stable) useful for the present invention. In various embodiments, the transfection of the second circular DNA molecule may be carried out first, in order to obtain cells exogenously expressing the DDX3 helicase, that are later used for a second transfection of the first circular DNA molecule to obtain double transfectants useful for the present invention.

The method disclosed herein may further comprises the step of cultivating or culturing the transfected host cell. Methods for culturing the transfected host cells are well-known in the art and can be routinely applied by those skilled in the art. Culturing conditions, reagents and mediums can be selected by those skilled in the art based on the host organism used by resorting to general knowledge and techniques known in the art. In various embodiments, the culturing step comprises incubating the transfected host cell in expression, suspension and/or growth media.

In various embodiments, the culturing step comprises passaging the transfected host cell one or more times in the presence or absence of selection pressure, whereby the transfected host cell is cultured in a media comprising an antibiotic or in an antibiotic-free media. In other words, the transfected host cell is, or is not, cultured and grown in a selection medium and is, or is not, cultured in the presence of a selection agent, such as an antibiotic.

In various embodiments, the host cell may be an eukaryotic host cell that is adherent or non-adherent. That is, the eukaryotic host cell may be adherent cells that naturally adhere to a solid substrate, or may be non-adherent cells that may be maintained as cells in a suspension of freely growing cells by cultivation in an appropriate cell culture system. In various embodiments, where the host cell is a non-adherent host cell, the transfection step may comprise adding a mix of Lipofectamine and the circular DNA molecule(s) to the host cells and incubated overnight under antibiotics free growth medium. In various embodiments, where the host cell is an adherent host cell, the adherent host cell may be initially grown to a desired confluence level (e.g. 70-90%) and then adding a mix of Lipofectamine and the circular DNA molecule(s) to the host cells and incubated under antibiotics free growth medium.

In various embodiments, the method further comprises identifying a reporter cell into which the circular DNA molecule(s) has been successfully introduced. In this regard, the successful introduction may be confirmed, and reporter cell identified, using any well-known technique and method based on the reporter gene, for example, PCR or Southern blotting to detect the circular DNA molecule in the cell; qRT-PCR or Northern blotting to confirm mRNA transcription of circular DNA molecule-encoded genes; Western blotting or immunofluorescence to detect the circular DNA molecule-encoded protein; Reporter gene assays (e.g., luciferase, GFP, B-gal) to confirm functional expression; Fluorescence microscopy or flow cytometry to visualize or quantify reporter gene expression; and/or antibiotic selection or PCR screening for stable transfections.

In various embodiments, the method may further comprise confirming if the reporter gene introduced into the cell is being actively translated into its corresponding protein. This may be carried out using the puromycin incorporation assay, or other similar well-known methods in the art.

In various embodiments, the method further comprises isolating the identified reporter cell to obtain the reporter cell for use in the methods further described herein. In various embodiments, the reporter cell may be isolated and expanded to generate a homogenous population of the reporter cells.

Accordingly, there is also provided a reporter cell obtained by the method of production disclosed herein. The reporter cell obtained from the method disclosed herein may be isolated and expanded in a cell culture medium suitable for expansion of the reporter cell. Accordingly, there is also provided a reporter cell culture comprising a plurality of reporter cells obtained from the method of production disclosed herein.

### Methods for Assessing the Activity of DDX3 Helicase

As will be appreciated, the reporter cell or cell culture line may then be used in the following described methods or may be generally used for assessing the activity of a DDX3 helicase, as described herein. Such uses thus also form part of the present invention.

All embodiments disclosed above in relation to the reporter cell and method of production similarly apply to the methods of use and vice versa.

In various embodiments, the method for assessing the activity of a DDX3 helicase, may comprise the following steps:
providing a reporter cell disclosed herein;
culturing the reporter cell under conditions that allow expression of the reporter gene to produce a reporter gene product, and exogenous or transient expression of the DDX3 helicase;
detecting the presence/absence, optionally quantity, of a detectable signal emitted by the reporter gene product to obtain a readout that is indicative of the activity of the DDX3 helicase.

In various embodiments, prior to the culturing step, the method may further comprise introducing a second circular DNA molecule into the reporter cell at suitable conditions to induce uptake of the second circular DNA molecule into the cell, wherein the second circular DNA molecule comprises a nucleotide sequence encoding the DDX3 helicase, and one or more regulatory elements capable of controlling and/or enhancing transient or exogenous expression of the DDX3 helicase whose activity is to be assessed.

Methods for culturing the reporter cells are well-known in the art and can be routinely applied by those skilled in the art. Culturing conditions, reagents and mediums can be selected by those skilled in the art based on the host organism used by resorting to general knowledge and techniques known in the art. In various embodiments, the culturing step comprises incubating the reporter cell in expression, suspension and/or growth media to allow expression of the reporter gene and exogenous or transient expression of the DDX3 helicase. In particular, this step may comprise inducing the expression of the DDX3 helicase in the reporter cell to facilitate the expression of the reporter gene and subsequent emission of a detectable signal.

In various embodiments, the signal emitted by the reporter gene product upon expression of said reporter gene may be detected through a variety of techniques and methods well-known in the art depending on the nature of the reporter gene. The reporter gene may emit fluorescent, luminescent, colorimetric, or other biochemical signals, and the detection methods will vary accordingly. The detection method of the emitted signal may include but is not limited to Fluorescence Microscopy, Luminescence Assays, B-Galactosidase Staining, Colorimetric Assays (Absorbance-Based Detection), Chemiluminescence Assay, Quantitative PCR (qPCR) for Reporter mRNA Expression, Western Blotting or ELISA for Reporter Protein Detection, or Functional Assays (Reporter-Linked Activity).

In various embodiments, the presence, and/or quantified level, of the detectable signal emitted by the reporter gene product indicates that the exogenously or transiently expressed DDX3 helicase is functionally active and has unwound the secondary structure of the 5′-UTR facilitating expression of the reporter gene and emission of the detectable signal.

In various embodiments, where the reporter gene is a luciferase reporter gene, the emitted signal from the gene product is a luminescence signal that may be detected and measured by a Luminescence Assay (Luminometer) that quantitatively measures the amount of luminescence produced by the reporter gene product. This assay includes the lysing of the reporter cells expressing the reporter gene, and adding a luciferase substrate (e.g., luciferin). The enzyme catalyses a reaction that emits light, which is detected and quantified using a luminometer to provide a readout that may be used for subsequent analysis.

The intensity of luminescence emitted by the luciferase gene product is proportional to the amount of the luciferase gene product produced. Thus, given that the expression of the luciferase gene is dependent upon the presence in the reporter cell of a functionally active DDX3 helicase to unwind the 5′-UTR, the luminescence signal can be used to infer the activity of the DDX3 helicase. That is, the intensity of the luminescence emitted by the luciferase gene product is directly proportional to the activity of the DDX3 helicase. Luminescence can be measured over time to assess the rate of activity of the DDX3 helicase. Accordingly, the luminescence emitted by a luciferase reporter gene may be qualitatively and/or quantitatively measured to provide a qualitative and/or quantitative readout of the activity of the DDX3 helicase.

In various embodiments, the detection of the emitted luminescence signal indicates that the exogenously or transiently expressed DDX3 helicase is functionally active and has unwound a secondary structure of the 5′-UTR facilitating the expression of the luciferase reporter gene to produce the luciferase enzyme and emission of the luminescence signal. In this regard, the emitted luminescence signal by the reporter gene product is proportional to the expression level of the luciferase reporter gene. Thus, a quantitative readout of the emitted luminescence signal is proportional to the expression level of the luciferase reporter gene and directly correlates to the activity of the DDX3 helicase. In various embodiments, a relative expression level of the luciferase reporter gene may be obtained by comparing to a control and normalizing.

In various embodiments, the method may further comprise analysing the detected signal level (i.e. the emitted signal readout). The analysis step may comprise comparing the detected signal level to a reference signal level, wherein a differential signal level, may be further indicative of the activity of the DDX3 helicase.

In various embodiments, the reference signal level may be derived from a reference cell or control cell. The term “reference cell”, “control cell,” or the like as used herein, refers to a cell, standard, or level that is used for comparison purposes. In various embodiments, the reference or control signal level may be derived from a control cell that comprises a reporter expression construct disclosed herein without inclusion of the 5′-UTR, and or a control cell that has endogenous expression of the DDX3 helicase (i.e. sgCTL).

As will be appreciated, the term “differential signal level” refers to a significant variation in the expression levels of the reporter gene and the corresponding emitted signal level of the gene product, relative to a reference/control signal level. Subsequently, the expression of the reporter gene and related detectable signal level may be identified as being upregulated (increased) or downregulated (decreased) relative to the reference/control signal level. Statistically significant differences may be determined by applying statistical tests well-known to those skilled in the art, for example, t-test, one-way ANOVA, or two-way ANOVA using appropriate software tools.

Accordingly, the step of analysing may comprise analysis of the differential signal level using methods readily known to those skilled in the art, which may typically involve normalization, statistical testing, and interpretation of results to assess and determine the activity of the DDX3 helicase.

Standard normalization methods suitable to each type of detection method and assay may, or may not, be employed. In various embodiments, the method includes normalizing the reporter gene product-emitted signal. For example, luminescence data may be normalized to controls, total protein concentration, or cell number to ensure the results reflect enzyme activity rather than differences in transfection efficiency or cell viability. Normalization is the process of adjusting data to account for variations or baseline factors (i.e. basal activity) that might affect the output. When normalizing enzyme activity or detectable signals, the stimulated or experimental state may be compared to the basal activity. This can help control for background signals or noise and ensure that the results reflect true changes in enzyme activity.

There is also provided a cell-based reporter system for assessing the activity of a DDX3 helicase, comprising: the reporter cell disclosed herein; and a means for detecting, and optionally quantifying, a detectable signal emitted by the reporter gene product, wherein an assessment of the activity of the DDX3 helicase is based on the presence/absence, optionally quantification, of the detectable signal emitted by the reporter gene product in the reporter cell, and wherein the presence, and optional quantity, of the detectable signal, correlates with the activity of the DDX3 helicase.

In various embodiments, the means for detecting may comprise one or more techniques and methods well-known in the art depending on the nature of the reporter gene and emitted signal to be detected. In various embodiments, the means for detecting may comprise one or more methods selected from Fluorescence Microscopy, Luminescence Assays, B-Galactosidase Staining, Colorimetric Assays (Absorbance-Based Detection), Chemiluminescence Assay, Quantitative PCR (qPCR) for Reporter mRNA Expression, Western Blotting or ELISA for Reporter Protein Detection, or Functional Assays (Reporter-Linked Activity). In various embodiments, the means for detecting may comprise a Luminescence Assay.

### Methods of Screening Candidate Agents for Modulating DDX3 Helicase Activity

The developed reporter cell disclosed herein, and method of assessing the activity of DDX3 helicases, can be applied to screen agents targeting DDX3 helicases in living cells and study their functional roles in healthy and disease states.

Accordingly, there is also provided a method for developing or identifying agents capable of modulating the activity of DDX3 helicase, the method comprising:
providing a reporter cell disclosed herein, wherein the reporter cell exogenously or transiently expresses a functionally active DDX3 helicase;
administering a candidate agent to the reporter cell;
measuring a detectable signal emitted by a reporter gene product;
comparing the measured detectable signal to a control,
wherein the candidate agent is identified as capable of modulating the activity of the DDX3 helicase, if the measured detectable signal of the reporter gene product is higher or lower relative to the control.

The term “modulating” refers to the process by which the agent alters or influences the activity of the DDX3 helicase either by increasing (enhancing) or decreasing (inhibiting) the DDX3 helicases functional ability. Modulation can occur through direct interaction with the DDX3 helicase or by affecting the regulatory mechanisms controlling expression or activity of the DDX3 helicase.

In various embodiments, a decrease of the measured detectable signal of the reporter gene product relative to the control is indicative of the candidate agent having inhibitory activity of DDX3 helicase, or an increase of the measured detectable signal of the reporter gene product relative to the control is indicative of the candidate agent having stimulatory activity of DDX3 helicase.

In various embodiments, the “control” refers to a measured detectable signal of the reporter gene product in the absence of the candidate agent (i.e. an untreated reporter cell without the administering step). Accordingly, the method comprises comparing the DDX3 helicase activity in the presence and absence of the candidate agent to assess its inhibitory or stimulatory effect on DDX3 activity. In other words, the outcome of the comparing step may result in identifying i) an alteration (differential level) in the detectable signal of the reporter gene product in the presence of the candidate agent relative to the detectable signal of the reporter gene product in the absence of the candidate agent, indicating that the candidate agent modulates DDX3 helicase activity; or (ii) no alteration in the detectable signal of the reporter gene product in the presence of the candidate agent relative to the detectable signal of the reporter gene product in the absence of the candidate agent, indicating that the candidate agent does not modulate DDX3 helicase activity.

In various embodiments, the alteration or differential in measured detectable signal following the comparison may further be compared to a threshold value or level. A threshold value or level may be associated with a statistic, whereby a threshold value or level is a value or level above or below which the alteration/difference in the measured detectable signal is assigned significance (i.e. via a statistical method), for example a p-value <0.05 may be considered as statistically significant.

In various embodiments, the method may further comprise culturing the reporter cell under conditions that allow expression of the reporter gene and exogenous or transient expression of the functionally active DDX3 helicase. In various embodiments, this culturing step may include inducing the exogenous or transient expression of the functionally active DDX3 helicase, prior to or after the candidate agent has been administered to the reporter cell.

The term “administering” and variations of that term including “administer” and “administration”, includes contacting, applying, delivering or providing an agent, compound or composition (i.e. candidate agent) to the reporter cell by any appropriate means. The method may be an in vitro, or ex vivo method of use.

The term “candidate agent” refers to an agent for which there is some evidence of potential therapeutic efficacy. The term candidate compound refers to a compound that may or may not have been previously evaluated for any therapeutic application, particularly in the context of a disease or condition involving aberrant DDX3 helicase activity. Any candidate agent or candidate compound may be implemented as the “agent” for achieving a particular physiological effect. In various embodiments, a candidate agent is a small molecule, protein (peptide or antibody), or nucleic acid. Consequently, candidate agents may also be a small molecule, protein, or nucleic acid. Such agents and compounds may be in a pharmaceutically acceptable formulation. As will be appreciated, the increases or decrease of the detected reporter gene signal (e.g. luminescence signal) relative to a control following administration of the candidate agent will characterize the candidate agent as being an inhibitor or stimulator of DDX3 helicase activity.

In various embodiments, the candidate agent may be selected from a virtual chemical library. As used herein, the term “library” refers to a plurality of compounds. A library can be a combinatorial library, e.g., a collection of compounds synthesized using combinatorial chemistry techniques, or a collection of unique chemicals of low molecular weight (less than 1000 daltons) that each occupy a unique three-dimensional space.

All embodiments disclosed above in relation to the reporter cell and methods similarly apply to the screening method and vice versa.

### EXAMPLES

### Materials and Methods

Cell culture: 293T cells (ATCC #CRL-3216) were cultured in Dulbecco's modified Eagle's medium (DMEM) supplemented with 10% heat-inactivated fetal bovine serum (HI-FBS). DDX3X WT and DDX3X R475C U2932 cells were cultured in Roswell Park Memorial Institute (RPMI) 1640 medium supplemented with 10% HI-FBS. Methods for generating the DDX3X mutant U2932 cells have been described previously [8]. Cells were maintained at 37° C. in 5% CO2 incubator. Cells underwent routine mycoplasma testing with MycoGuard™ Mycoplasma PCR Detection Kit (Genecopoeia #MP004) and were verified free of mycoplasma prior to the commencement of experiments.

Cell transfection: 293T cells were seeded in 12 well plate (1.5×105 cells per well) a day prior to transfection. Cells were transfected with 1 μg of plasmids using Lipofectamine 2000 (ThermoFisher Scientific #11668019) as per manufacturer's recommendations.

CRISPR-Cas9 knockout: Plasmids expressing Cas9 and guide RNA [pSpCas9 (BB)-2A-GFP (PX458), Addgene #48138] were used to create stable deletion of DDX3X in 293T cells. The sgRNAs target sequences (5′→3′) were: DDX3X sg1 (CGTGGACGGAGTGATTACGA (SEQ ID NO: 20)), DDX3X sg2 (CGGAGTGATTACGATGGCAT (SEQ ID NO:21)), HBEGF sg10 (GCAAATATGTGAAGGAGCTC (SEQ ID NO:22)), and Cas9 control (GTGTAGTTCGACCATTCGTG (SEQ ID NO:23)). To enrich DDX3X knockout (KO) 293T cells, we selected for KO cells using diphtheria toxin (DT, Sigma #D0564). Briefly, WT unmodified 293T cells were co-transfected with plasmids expressing sgRNA against DDX3X and HBEGF and subsequently selected with DT (20 ng/mL) until all the WT kill control cells have been killed. Single cell clones were selected 298 to establish complete DDX3X KO population. KO efficiency was tested via Western immunoblot and further confirmed using PCR against genomic DNA at the targeted region.

Plasmids: All expression plasmids for transient expression were cloned into pLVX vector backbone using standard restriction cloning. Site-directed mutagenesis was performed using Q5® Site-Directed Mutagenesis Kit Protocol (NEB #E0554) to generate pathogenic DDX3X mutants. Sanger sequencing was performed to confirm successful mutation at the desired site. The reporter construct used in our study was made by Vectorbuilder with vector ID VB220922-1486nnk (pcDNA3.1 (+)-5′-UTR_RAC1: luciferase) and VB220922-1483jcb (pcDNA3.1(+)-5′-UTR_DVL2:luciferase).

Luciferase assay: Cells were lysed in luciferase cell culture lysis buffer (Promega #E1500) before measuring bioluminescence in a clear bottom 96-well plate using luciferase assay system (Promega #E4030) as per manufacturer's instructions. Relative luciferase activity was determined by subtracting luminescence values to blank and normalizing to untreated samples. Cycloheximide (Sigma #01810) was used as a positive control for translation inhibition at 1 μg/mL and RK-33 (Selleck #S8246) was used as a positive control for chemical inhibition of DDX3 at 3 μM.

Puromycin incorporation assay: Prior to pulsing the cells with puromycin, positive control well was pre-treated with cycloheximide (1 μg/mL) for 3 h to inhibit translation. Subsequently, all the wells were subjected to puromycin (Gibco #A1113803) at 1 μg/mL for 30 min except for negative control well before harvesting of cell lysate. Global protein synthesis was determined by normalizing puromycin incorporation bands against ponceau S staining.

Western immunoblotting: Cells were washed with ice-cold phosphate buffer saline before lysing as described earlier. Extracted proteins were quantified via Bradford Assay (Bio-Rad #5000001) and equal amount of cell lysates (20-40 μg) were resolved on 8-20% gradient gel. Proteins were then transferred from the gel onto an activated PVDF membrane using Trans-Blot Turbo Transfer System (Bio-Rad #1704156). Membrane was incubated in 3% skim milk in tris buffer saline 0.1% Tween-20 (TBS-T) for 30 minutes at room temperature before incubating with the relevant primary antibodies overnight at 4° C. with gentle shaking. The following primary antibodies were used: DDX3X (Bethyl Laboratories #A300-474A), GAPDH (Millipore #AB2302), a-tubulin (Sigma #T5168, anti-Flag (Sigma #F1804) and anti-Puromycin (Sigma #MABE343). Primary antibodies were used at a concentration around 250 ng/ml. The next day, the membrane was washed 3 times in TBST before incubating with the appropriate HRP secondary antibodies. For secondary antibodies, anti-rabbit HRP conjugate (Jackson Immunoresearch #111-035-144) or anti-mouse HRP conjugate (Jackson Immunoresearch #115-035-166) were used when appropriate (1:7000 dilution). Blots were visualized on the ChemiDoc Imaging Systems (Bio-Rad) using SuperSignal™ West Pico PLUS Chemiluminescent Substrate (ThermoFisher Scientific #34580), Clarity Western ECL Substrate (Bio-Rad #1705061) or Clarity Max Western ECL Substrate (Bio-Rad #1705062) when appropriate.

Data analysis: Statistical analysis was performed as described in the figure legends with either t-tests (for comparison between two groups), one-way ANOVA (for comparison between >2 experimental groups) or two-way ANOVA (multiple comparison within experimental groups) using GraphPad Prism 9.3.0 software. P values<0.05 were considered statistically significant.

TABLE 2
DDX3 Sequences

| | Description | |
|---|---|---|
| SEQ ID | (Accession | |
| NO: | Number) | Amino acid Sequence |
| 2 | Human | MSHVAVENALGLDQQFAGLDLNSSDNQSGGSTASKGRYIPPHLR |
| | DDX3X | NREATKGFYDKDSSGWSSSKDKDAYSSFGSRSDSRGKSSFFSDR |
| | UniProt: | GSGSRGRFDDRGRSDYDGIGSRGDRSGFGKFERGGNSRWCDKSD |
| | O00571 | EDDWSKPLPPSERLEQELFSGGNTGINFEKYDDIPVEATGNNCP |
| | | PHIESFSDVEMGEIIMGNIELTRYTRPTPVQKHAIPIIKEKRDL |
| | | MACAQTGSGKTAAFLLPILSQIYSDGPGEALRAMKENGRYGRRK |
| | | QYPISLVLAPTRELAVQIYEEARKFSYRSRVRPCVVYGGADIGQ |
| | | QIRDLERGCHLLVATPGRLVDMMERGKIGLDFCKYLVLDEADRM |
| | | LDMGFEPQIRRIVEQDTMPPKGVRHTMMFSATFPKEIQMLARDF |
| | | LDEYIFLAVGRVGSTSENITQKWWWVEESDKRSFLLDLLNATGK |
| | | DSLTLVFVETKKGADSLEDFLYHEGYACTSIHGDRSQRDREEAL |
| | | HQFRSGKSPILVATAVAARGLDISNVKHVINFDLPSDIEEYVHR |
| | | IGRTGRVGNLGLATSFFNERNINITKDLLDLLVEAKQEVPSWLE |
| | | NMAYEHHYKGSSRGRSKSSRFSGGFGARDYRQSSGASSSSFSSS |
| | | RASSSRSGGGGHGSSRGFGGGGYGGFYNSDGYGGNYNSQGVDWW |
| | | GN |
| 3 | Human | MSHVVVKNDPELDQQLANLDLNSEKQSGGASTASKGRYIPPHLR |
| | DDX3Y | NREASKGFHDKDSSGWSCSKDKDAYSSFGSRDSRGKPGYFSERG |
| | UniProt: | SGSRGRFDDRGRSDYDGIGNRERPGFGRFERSGHSRWCDKSVED |
| | O15523 | DWSKPLPPSERLEQELFSGGNTGINFEKYDDIPVEATGSNCPPH |
| | | IENFSDIDMGEIIMGNIELTRYTRPTPVQKHAIPIIKGKRDLMA |
| | | CAQTGSGKTAAFLLPILSQIYTDGPGEALKAVKENGRYGRRKQY |
| | | PISLVLAPTRELAVQIYEEARKFSYRSRVRPCVVYGGADIGQQI |
| | | RDLERGCHLLVATPGRLVDMMERGKIGLDFCKYLVLDEADRMLD |
| | | MGFEPQIRRIVEQDTMPPKGVRHTMMFSATFPKEIQMLARDFLD |
| | | EYIFLAVGRVGSTSENITQKWWWVEDLDKRSFLLDILGATGSDS |
| | | LTLVFVETKKGADSLEDFLYHEGYACTSIHGDRSQRDREEALHQ |
| | | FRSGKSPILVATAVAARGLDISNVRHVINFDLPSDIEEYVHRIG |
| | | RTGRVGNLGLATSFFNEKNMNITKDLLDLLVEAKQEVPSWLENM |
| | | AYEHHYKGGSRGRSKSNRFSGGFGARDYRQSSGSSSSGFGASRG |
| | | SSSRSGGGGYGNSRGFGGGGYGGFYNSDGYGGNYNSQGVDWWGN |
| 4 | Mouse Ddx3x | MSHVAVENALGLDQQFAGLDLNSSDNQSGGSTASKGRYIPPHLR |
| | UniProt: | NREATKGFYDKDSSGWSSSKDKDAYSSFGSRGDSRGKSSFFGDR |
| | Q62167 | GSGSRGRFDDRGRGDYDGIGGRGDRSGFGKFERGGNSRWCDKSD |
| | | EDDWSKPLPPSERLEQELFSGGNTGINFEKYDDIPVEATGNNCP |
| | | PHIESFSDVEMGEIIMGNIELTRYTRPTPVQKHAIPIIKEKRDL |
| | | MACAQTGSGKTAAFLLPILSQIYADGPGEALRAMKENGRYGRRK |
| | | QYPISLVLAPTRELAVQIYEEARKFSYRSRVRPCVVYGGAEIGQ |
| | | QIRDLERGCHLLVATPGRLVDMMERGKIGLDFCKYLVLDEADRM |
| | | LDMGFEPQIRRIVEQDTMPPKGVRHTMMFSATFPKEIQMLARDF |
| | | LDEYIFLAVGRVGSTSENITQKVVWVEEIDKRSFLLDLLNATGK |
| | | DSLTLVFVETKKGADSLEDFLYHEGYACTSIHGDRSQRDREEAL |
| | | HQFRSGKSPILVATAVAARGLDISNVKHVINFDLPSDIEEYVHR |
| | | IGRTGRVGNLGLATSFFNERNINITKDLLDLLVEAKQEVPSWLE |
| | | NMAFEHHYKGSSRGRSKSSRFSGGFGARDYRQSSGASSSSFSSS |
| | | RASSSRSGGGGHGGSRGFGGGGYGGFYNSDGYGGNYNSQGVDWW |
| | | GN |
| 5 | Mouse Ddx3y | MSQVAAESTAGLDQQFVGLDLKSSDNQNGGGNTESKGRYIPPHL |
| | UniProt: | RNRETSKGVCDKDSSGWSCSKDKDAYSSFGSRDSRGKPNYFSDR |
| | Q62095 | GSGSRGRFDDHGRNDYDGIGGRDRTGFGKFERSGHSRWSDRSDE |
| | | DDWSKPLPPSERLEQELFSGGNTGINFEKYDDIPVEATGNNCPP |
| | | HIENFSDIEMGEIIMGNIELTRYTRPTPVQKHAIPIIKEKRDLM |
| | | ACAQTGSGKTAAFLLPILSQIYTDGPGEALKAMKENGRYGRRKQ |
| | | YPISLVLAPTRELAVQIYEEARKFSYRSRVRPCVVYGGADTVQQ |
| | | IRDLERGCHLLVATPGRLVDMMERGKIGLDFCKYLVLDEADRML |
| | | DMGFEPQIRRIVEQDTMPPKGVRHTMMFSATFPKEIQMLARDFL |
| | | DEYIFLAVGRVGSTSENITQKVVWVEELDKRSFLLDLLNATGKD |
| | | SLTLVFVETKKGADSLENFLFQERYACTSIHGDRSQKDREEALH |
| | | QFRSGRKPILVATAVAARGLDISNVKHVINFDLPSDIEEYVHRI |
| | | GRTGRVGNLGLATSFFNERNLNITKDLLDLLVEAKQEVPSWLES |
| | | MAYEHHYKGSSRGRSKSRFSGGFGARDYRQSSGSANAGENSNRA |
| | | NSSRSSGSSHNRGFGGGGYGGFYNNDGYGGNYNSQAVDWWGN |

TABLE 3
Reporter Expression Construct Sequences

| | Description | |
|---|---|---|
| SEQ ID | (Accession | |
| NO: | Number) | Nucleotide Sequence |
| 6 | CMV promoter | GTGATGCGGTTTTGGCAGTACATCAATGGGCGTGGATAGCGGT |
| | sequence | TTGACTCACGGGGATTTCCAAGTCTCCACCCCATTGACGTCAA |
| | | TGGGAGTTTGTTTTGGCACCAAAATCAACGGGACTTTCCAAAA |
| | | TGTCGTAACAACTCCGCCCCATTGACGCAAATGGGCGGTAGGC |
| | | GTGTACGGTGGGAGGTCTATATAAGCAGAGCT |
| 7 | Fluc | ATGGAAGACGCCAAAAACATAAAGAAAGGCCCGGCGCCATTCT |
| | | ATCCGCTAGAGGATGGAACCGCTGGAGAGCAACTGCATAAGGC |
| | | TATGAAGAGATACGCCCTGGTTCCTGGAACAATTGCTTTTACA |
| | | GATGCACATATCGAGGTGAACATCACGTACGCGGAATACTTCG |
| | | AAATGTCCGTTCGGTTGGCAGAAGCTATGAAACGATATGGGCT |
| | | GAATACAAATCACAGAATCGTCGTATGCAGTGAAAACTCTCTT |
| | | CAATTCTTTATGCCGGTGTTGGGCGCGTTATTTATCGGAGTTG |
| | | CAGTTGCGCCCGCGAACGACATTTATAATGAACGTGAATTGCT |
| | | CAACAGTATGAACATTTCGCAGCCTACCGTAGTGTTTGTTTCC |
| | | AAAAAGGGGTTGCAAAAAATTTTGAACGTGCAAAAAAAATTAC |
| | | CAATAATCCAGAAAATTATTATCATGGATTCTAAAACGGATTA |
| | | CCAGGGATTTCAGTCGATGTACACGTTCGTCACATCTCATCTA |
| | | CCTCCCGGTTTTAATGAATACGATTTTGTACCAGAGTCCTTTG |
| | | ATCGTGACAAAACAATTGCACTGATAATGAACTCCTCTGGATC |
| | | TACTGGGTTACCTAAGGGTGTGGCCCTTCCGCATAGAACTGCC |
| | | TGCGTCAGATTCTCGCATGCCAGAGATCCTATTTTTGGCAATC |
| | | AAATCATTCCGGATACTGCGATTTTAAGTGTTGTTCCATTCCA |
| | | TCACGGTTTTGGAATGTTTACTACACTCGGATATTTGATATGT |
| | | GGATTTCGAGTCGTCTTAATGTATAGATTTGAAGAAGAGCTGT |
| | | TTTTACGATCCCTTCAGGATTACAAAATTCAAAGTGCGTTGCT |
| | | AGTACCAACCCTATTTTCATTCTTCGCCAAAAGCACTCTGATT |
| | | GACAAATACGATTTATCTAATTTACACGAAATTGCTTCTGGGG |
| | | GCGCACCTCTTTOGAAAGAAGTCGGGGAAGCGGTTGCAAAACG |
| | | CTTCCATCTTCCAGGGATACGACAAGGATATGGGCTCACTGAG |
| | | ACTACATCAGCTATTCTGATTACACCCGAGGGGGATGATAAAC |
| | | CGGGCGCGGTCGGTAAAGTTGTTCCATTTTTTGAAGCGAAGGT |
| | | TGTGGATCTGGATACCGGGAAAACGCTGGGCGTTAATCAGAGA |
| | | GGCGAATTATGTGTCAGAGGACCTATGATTATGTCCGGTTATG |
| | | TAAACAATCCGGAAGCGACCAACGCCTTGATTGACAAGGATGG |
| | | ATGGCTACATTCTGGAGACATAGCTTACTGGGACGAAGACGAA |
| | | CACTTCTTCATAGTTGACCGCTTGAAGTCTTTAATTAAATACA |
| | | AAGGATACCAGGTGGCCCCCGCTGAATTGGAGTCGATATTGTT |
| | | ACAACACCCCAACATCTTCGACGCGGGCGTGGCAGGTCTTCCC |
| | | GACGATGACGCCGGTGAACTTCCCGCCGCCGTTGTTGTTTTGG |
| | | AGCACGGAAAGACGATGACGGAAAAAGAGATCGTGGATTACGT |
| | | CGCCAGTCAAGTAACAACCGCGAAAAAGTTGCGCGGAGGAGTT |
| | | GTGTTTGTGGACGAAGTACCGAAAGGTCTTACCGGAAAACTCG |
| | | ACGCAAGAAAAATCAGAGAGATCCTCATAAAGGCCAAGAAGGG |
| | | CGGAAAGTCCAAATTGTAA |
| 8 | IRES | CCCCTCTCCCTCCCCCCCCCCTAACGTTACTGGCCGAAGCCGC |
| | | TTGGAATAAGGCCGGTGTGCGTTTGTCTATATGTTATTTTCCA |
| | | CCATATTGCCGTCTTTTGGCAATGTGAGGGCCCGGAAACCTGG |
| | | CCCTGTCTTCTTGACGAGCATTCCTAGGGGTCTTTCCCCTCTC |
| | | GCCAAAGGAATGCAAGGTCTGTTGAATGTCGTGAAGGAAGCAG |
| | | TTCCTCTGGAAGCTTCTTGAAGACAAACAACGTCTGTAGCGAC |
| | | CCTTTGCAGGCAGCGGAACCCCCCACCTGGCGACAGGTGCCTC |
| | | TGCGGCCAAAAGCCACGTGTATAAGATACACCTGCAAAGGCGG |
| | | CACAACCCCAGTGCCACGTTGTGAGTTGGATAGTTGTGGAAAG |
| | | AGTCAAATGGCTCTCCTCAAGCGTATTCAACAAGGGGCTGAAG |
| | | GATGCCCAGAAGGTACCCCATTGTATGGGATCTGATCTGGGGC |
| | | CTCGGTACACATGCTTTACATGTGTTTAGTCGAGGTTAAAAAA |
| | | ACGTCTAGGCCCCCCGAACCACGGGGACGTGGTTTTCCTTTGA |
| | | AAAACACGATGATAATATGGCCACAACC |
| 9 | AcGFP | ATGGTGAGCAAGGGCGAGGAGCTGTTCACCGGGGTGGTGCCCA |
| | | TCCTGGTCGAGCTGGACGGCGACGTAAACGGCCACAAGTTCAG |
| | | CGTGTCCGGCGAGGGCGAGGGCGATGCCACCTACGGCAAGCTG |
| | | ACCCTGAAGTTCATCTGCACCACCGGCAAGCTGCCCGTGCCCT |
| | | GGCCCACCCTCGTGACCACCCTGACCTACGGCGTGCAGTGCTT |
| | | CAGCCGCTACCCCGACCACATGAAGCAGCACGACTTCTTCAAG |
| | | TCCGCCATGCCCGAAGGCTACGTCCAGGAGCGCACCATCTTCT |
| | | TCAAGGACGACGGCAACTACAAGACCCGCGCCGAGGTGAAGTT |
| | | CGAGGGCGACACCCTGGTGAACCGCATCGAGCTGAAGGGCATC |
| | | GACTTCAAGGAGGACGGCAACATCCTGGGGCACAAGCTGGAGT |
| | | ACAACTACAACAGCCACAACGTCTATATCATGGCCGACAAGCA |
| | | GAAGAACGGCATCAAGGTGAACTTCAAGATCCGCCACAACATC |
| | | GAGGACGGCAGCGTGCAGCTCGCCGACCACTACCAGCAGAACA |
| | | CCCCCATCGGCGACGGCCCCGTGCTGCTGCCCGACAACCACTA |
| | | CCTGAGCACCCAGTCCGCCCTGAGCAAAGACCCCAACGAGAAG |
| | | CGCGATCACATGGTCCTGCTGGAGTTCGTGACCGCCGCCGGGA |
| | | TCACTCTCGGCATGGATGAGCTGTACAAGTGA |
| 10 | Blasticidin | ATGGCCAAGCCTTTGTCTCAAGAAGAATCCACCCTCATTGAAA |
| | S-resistance | GAGCAACGGCTACAATCAACAGCATCCCCATCTCTGAAGACTA |
| | gene | CAGCGTCGCCAGCGCAGCTCTCTCTAGCGACGGCCGCATCTTC |
| | | ACTGGTGTCAATGTATATCATTTTACTGGGGGACCTTGTGCAG |
| | | AACTCGTGGTGCTGGGCACTGCTGCTGCTGCGGCAGCTGGCAA |
| | | CCTGACTTGTATCGTCGCGATCGGAAATGAGAACAGGGGCATC |
| | | TTGAGCCCCTGCGGACGGTGCCGACAGGTGCTTCTCGATCTGC |
| | | ATCCTGGGATCAAAGCCATAGTGAAGGACAGTGATGGACAGCC |
| | | GACGGCAGTTGGGATTCGTGAATTGCTGCCCTCTGGTTATGTG |
| | | TGGGAGGGCTAA |

TABLE 4
Sequences of 5′-UTRs

| SEQ | Description | |
|---|---|---|
| ID | (Accession | |
| NO: | Number) | Nucleotide Sequence |
| 11 | control | GGTTTAGTGAACCGTCAGATCCGCTAGC |
| 12 | ATF5 | ATTTGGGAGAGTGCTGTGACTCATGCTGGACTCTAACCCACG |
| | (ENST00000595125) | AGGGTTTCTCAGAGTCAGCAGCTGGGGGATGAAGAAGTGAAA |
| | | AGTGACTGGCAGGAAATTCTGCAAGCAAGGAAAGGGAAAGAG |
| | | AAATGAACTGGTGCAGGTCTGCGGGAAGAGAATGAGGCTGGA |
| | | TCCTCAAAATCACAGGAGGAAGCAGGCCCAGACCTCAGAGGC |
| | | AGAAAGAGAAAGAAACCAGAGCTTAGAGTCAGGAGGAGGAAA |
| | | CCAGACCCCGGAGCCACAAGGAGAGGGCTGGATCCCCGGCTC |
| | | AGAGGGAAGAGTGTCGCCGCCTCTGCCTGCGTAGCCCCGGCC |
| | | ATGGCTCTGTAGCCTCGACCCCTTTGTGCCCCCGGCCCGTCT |
| | | CCGCGCTCACCACGCCTGCGCTCTCCGCTCCCACCTTCTTTC |
| | | TTCAGCCGAGGCCGCCGCCGCCTCTCCTTGCTGCAGCCATGG |
| | | AGTCTTCCACTTTCGCCTTGGTGCCTGTCTTCGCCCACCTGA |
| | | GCATCCTCCAGAGCCTCGTGCCAGCTGCTGGTGCAGCCTCTC |
| | | CTGTTGCCATCAGTGCCCAGCACCTGTGCTACAGCC |
| 13 | RPLP1 | TGGACACATAAGAGGCTGCGTATAGGCGCGAGAGCCCCTTTC |
| | (ENST00000260379) | CTCAGCTGCCGCCAAGGTGCTCGGTCCTTCCGAGGAAGCTAA |
| | | GGCTGCGTTGGGGTGAGGCCCTCACTTCATCCGGCGACTAGC |
| | | ACCGCGTCCGGCAGCGCCAGCCCTACACTCGCCCGCGCC |
| 14 | DVL2 short | TCGCACCCCGCGGCCCGCCCCCCGCCGCCACCCTCGCAGATC |
| | | CGTGCTTTTTCCCCTTTGCTTCTCTCCCGTACTGGGTCAGTC |
| | | CTGTCCGCGCTCGCGCGTCGGTTTGCGGGTGTGCGCAGGCGC |
| | | GGCAGGGGCCATTAGCCCTTTGGGTGGGCGGTGGAGCCCGGG |
| | | AGCGCGCGGGCGAGACC |
| 15 | DVL2_long | TTAAGTCACGTGACATGAGGAGAGGTGGGCGGGTACCTGGAG |
| | (ENST00000005340) | GAAGCTCGCGGCGTCGGTGGCGGTGGCGCGCGGCGGCCGCTG |
| | | AGACCGGGGCTTTGAGTCGCACCCCGCGGCCCGCCCCCCGCC |
| | | GCCACCCTCGCAGATCCGTGCTTTTTCCCCTTTGCTTCTCTC |
| | | CCGTACTGGGTCAGTCCTGTCCGCGCTCGCGCGTCGGTTTGC |
| | | GGGTGTGCGCAGGCGCGGCAGGGGCCATTAGCCCTTTGGGTG |
| | | GGCGGTGGAGCCCGGGAGCGCGCGGGCGAGACC |
| 16 | PRKRA | GCGAGGGGGCGTAGCCGGAGCTACGGCACCAAGGCTCCGCCC |
| | (ENST00000325748) | CCACCCTGCCTGCCCCCTCGCTGGAGCAACGCAAGCAGGAGG |
| | | CGGGGGAGTCGGAGGAGGTGGCGGCGCTGGAGCTCCTCCCGG |
| | | GGACCAGCGACCCGGGGAGCGAGCACGTCGCTCCGCACCGCT |
| | | CTTCCTCCAGCCGCTGAGCCGTCCCTTCTCGCC |
| 17 | RAC1 | AGTTTTCCTCAGCTTTGGGTGGTGGCCGCTGCCGGGCATCGG |
| | (ENST00000356142) | CTTCCAGTCCGCGGAGGGCGAGGCGGCGTGGACAGCGGCCCC |
| | | GGCACCCAGCGCCCCGCCGCCCGCAAGCCGCGCGCCCGTCCG |
| | | CCGCGCCCCGAGCCCGCCGCTTCCTATCTCAGCGCCCTGCCG |
| | | CCGCCGCCGCGGCCCAGCGAGCGGCCCTG |
| 18 | ODC1 | GACGTCGGCCCGCCGGCGCCCCACCAGCTCCGCGCGGGCCCG |
| | (ENST00000234111) | GGTTGGCCACCGCCGGGCCCCCGCCCCTCCCCCGGCGGTGTC |
| | | CCGGCCGGAACCGATCGTGGCTGGTTTGAGCTGGTGCGTCTC |
| | | CATGGCGACCCGCCGGTGCTATAAGTAGGGAGCGGCGTGCCG |
| | | TGGGGCTTTGTCAGTCCCTCCTGTAGCCGCCGCCGCCGCCGC |
| | | CCGCCGCCCCTCTGCCAGCAGCTCCGGCGCCACCTCGGGCCG |
| | | GCGTCTCCGGCGGGCGGGAGCCAGGCGCTGACGGGCGCGGCG |
| | | GGGGCGGCCGAGCGCTCCTGCGGCTGCGACTCAGGCTCCGGC |
| | | GTCTGCGCTTCCCCATGGGGCTGGCCTGCGGCGCCTGGGCGC |
| | | TCTGAGATTGTCACTGCTGTTCCAAGGGCACACGCAGAGGGA |
| | | TTTGGAATTCCTGGAGAGTTGCCTTTGTGAGAAGCTGGAAAT |
| | | ATTTCTTTCAATTCCATCTCTTAGTTTTCCATAGGAACATCA |
| | | AGAAATC |
| 19 | CCNE1 | GAGGGGCTGGGAGCCGCGGCGGGGCGGTGCGAGGGGGGCCGG |
| | (ENST00000262643) | GGCCGGTTCCGCGCGCAGGGATTTTAAATGTCCCGCTCTGAG |
| | | CCGGGCGCAGGAGCAGCCGGCGCGGCCGCCAGCGCGGTGTAG |
| | | GGGGCAGGCGCGGATCCCGCCACCGCCGCGCGCTCGGCCCGC |
| | | CGACTCCCGGCGCCGCCGCCGCCACTGCCGTCGCCGCCGCCG |
| | | CCTGCCGGGACTGGAGCGCGCCGTCCGCCGCGGACAAGACCC |
| | | TGGCCTCAGGCCGGAGCAGCCCCATC |

TABLE 5
Statistics of Reporter 5′-UTRs

| Reporter | GC | Nt | 75 nt region ΔG | Upstream start |
|---|---|---|---|---|
| 5′UTR | Content | length | min (kcal mol⁻¹) | sites (TISdb) |
| control | 66.7 | 9 | 0 | No |
| ATF5 | 59.8 | 582 | −28.2 | Yes |
| CCNE1 | 80.6 | 279 | −45.3 | No |
| RPLP1 | 66.1 | 165 | −33.7 | Yes |
| DVL2 (long) | 71.6 | 285 | −48.0 | Yes |
| DVL2 (short) | 73.0 | 185 | −40.3 | Yes |
| ODC1 | 69.5 | 511 | −45.4 | Yes |
| PRKRA | 74.6 | 201 | −40.1 | No |
| RAC1 | 79.2 | 197 | −48.3 | No |

### Results and Discussion

### Example 1: Construction of the ICD-Helicase Reporter Cell Systems

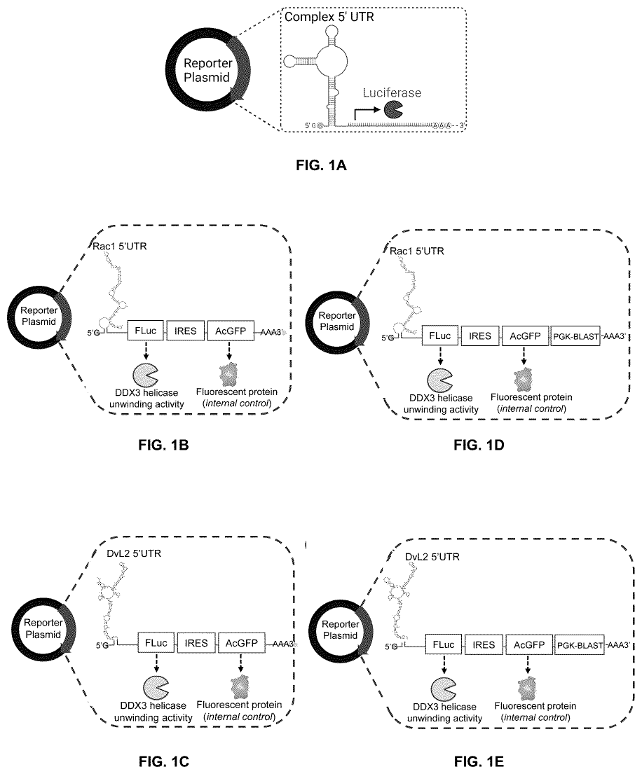

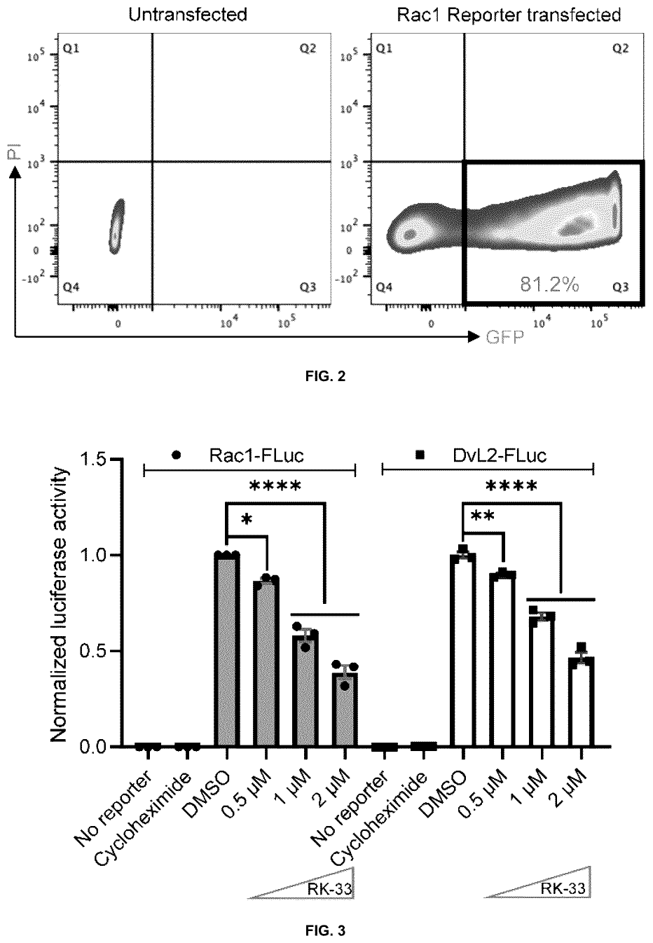

The helicase function of the DDX3X and DDX3Y proteins regulates mRNA translation by unwinding of the complex 5′ untranslated region (5′-UTR) secondary structure [12]. Two unique 5′-UTR structures were designed, Rac1 5′-UTR and DvL2 5′-UTR (i.e. short DVL2), known to be regulated by DDX3X and DDX3Y. The 5′-UTR segments were placed upstream of the luciferase gene (from the firefly Photinus pyralis) driven by a constitutively active human cytomegalovirus promoter (FIG. 1A-E). Luciferase emits bioluminescence signals proportional to its levels of expression, which served as a readout for helicase unwinding activity in ICD-Helicase assays. The reporter plasmids (Rac1-Fluc or DvL2-Fluc) were then transiently transfected into 293T cells. A transfection efficiency of more than 80% was obtained in all the experiments (FIG. 2). The selection of this cell line was for three main reasons. First, the 293T cell line originates from a female and does not express the Y chromosome homolog DDX3Y. Second, 293T cells can tolerate knock-out (KO) or nearly complete knock-down of DDX3X expression. Third, it is relatively easy to re-transduce wild-type (WT) or mutant plasmids in the KO/transgenic 293T cells. Both Rac1-Fluc and DvL2-Fluc reporter cells emitted luminescent signals that were significantly inhibited in the presence of RK-33, a non-specific DDX3 inhibitor [13] or the eukaryotic protein synthesis inhibitor cycloheximide, used as a control (FIG. 3, FIG. 8).

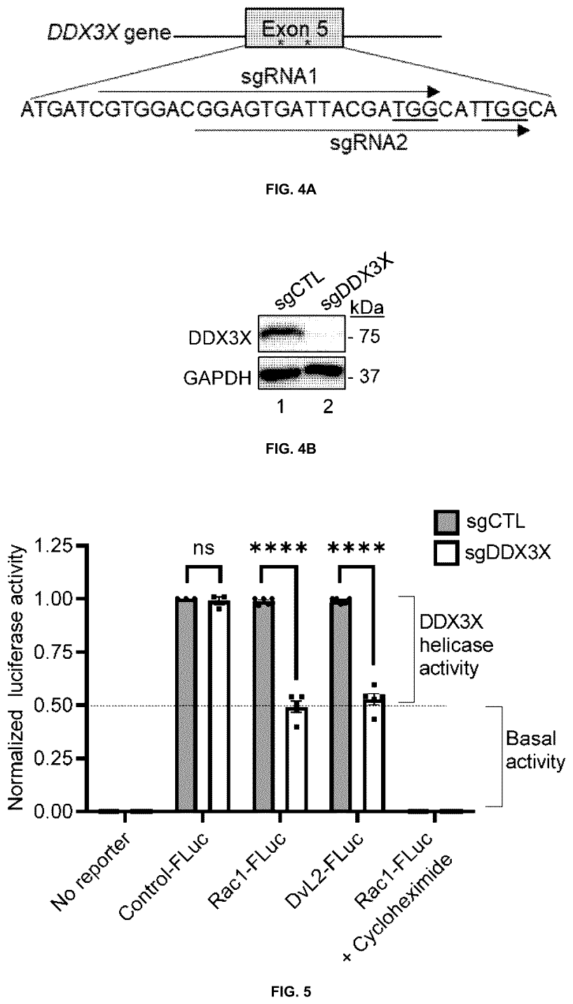

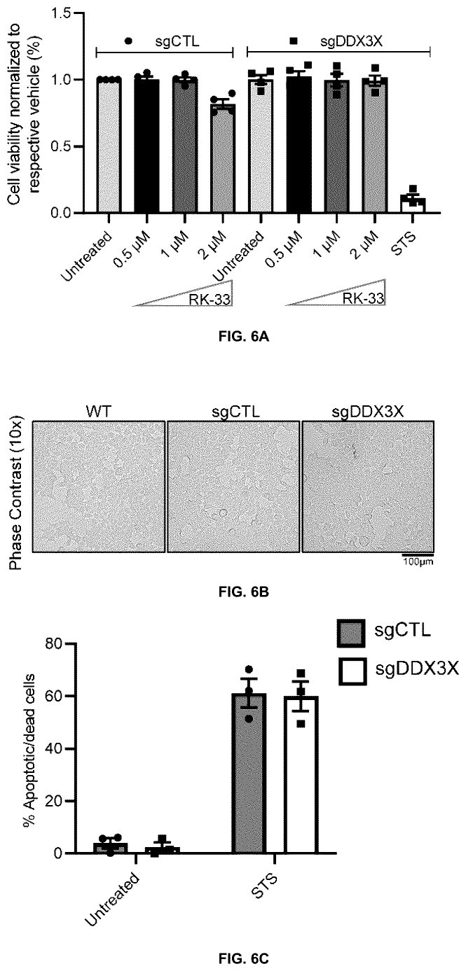

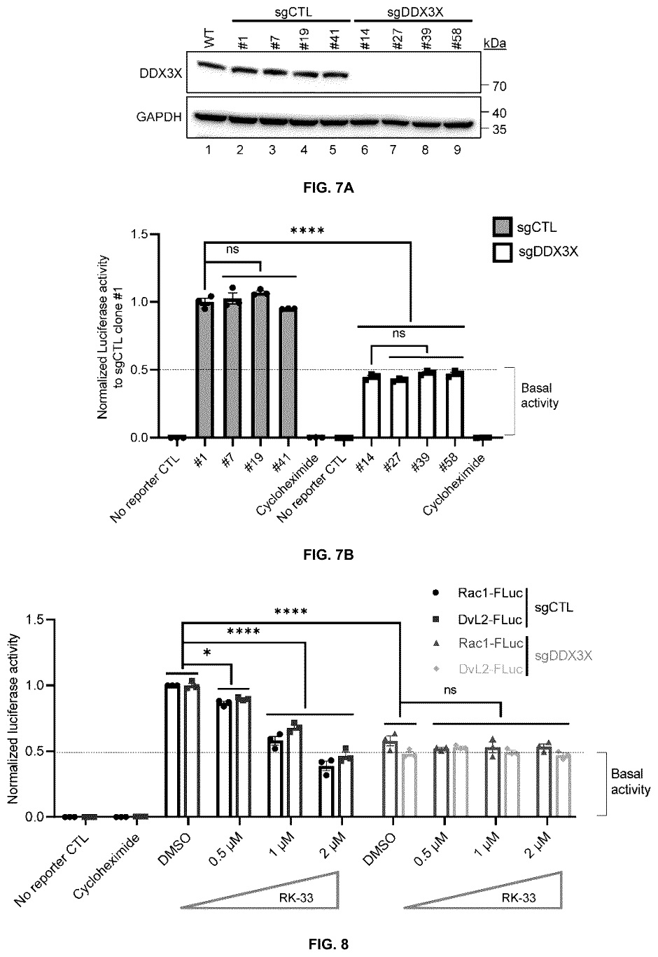

Since endogenous DDX3X in 293T cells can interfere with the exogenously expressed DDX3X or DDX3Y protein, DDX3X was knocked-out in these cells using CRISPR/Cas9 (sgDDX3X, FIG. 4A). The complete depletion of DDX3X protein in sgDDX3X 293T cells was confirmed by Western immunoblotting (FIG. 4B). As expected, the luciferase signal was significantly reduced in sgDDX3X Rac1-Fluc reporter cells compared to the control sgCTL cells (FIG. 5). Similar results were obtained with sgDDX3X DvL2-Fluc reporter cells (FIG. 5). Selective depletion of DDX3X by CRISPR Cas9 mediated knockout or DDX3 inhibition by RK-33 treatment was tolerated by 293T cells as there was no major change in cell viability or induction of apoptosis/cell death observed (FIG. 6A-6C) and the four other isogenic sgDDX3X clones that were generated (FIG. 7A-7B).

### Example 2: Cancer Associated DDX3X Mutants have Dysfunctional Helicase Activity

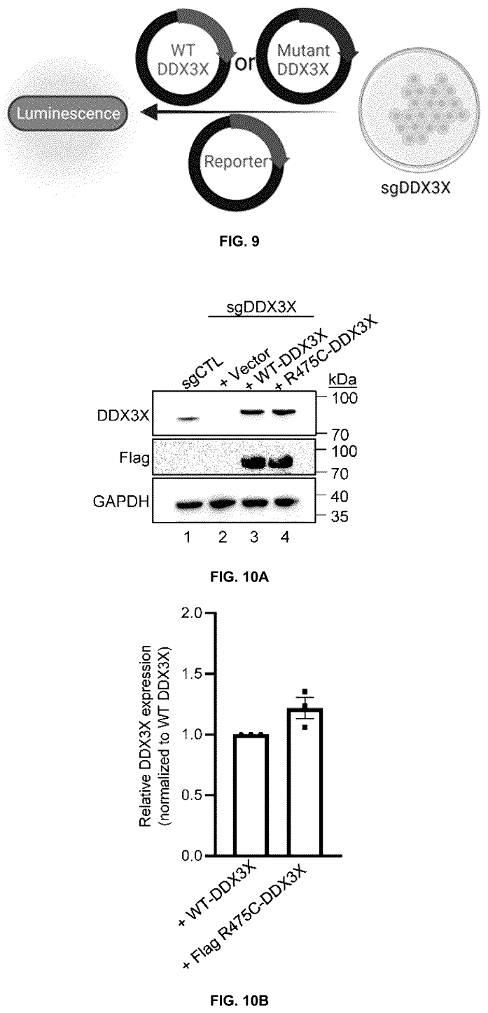

The helicase activity of exogenously expressed DDX3X and its mutants was determined. For this, either WT-DDX3X or mutant R475C-DDX3X plasmid was transfected in sgDDX3X 293T cells (FIG. 9, FIG. 10A-10B). Subsequently, cellular luciferase signals were measured using the reporter plasmid systems Rac1-Fluc and DvL2-Fluc. Results showed that reconstituting sgDDX3X in Rac1-Fluc or DvL2-Fluc 293T cells with WT-DDX3X rescued the ability of cells to unwind the 5′-UTR (FIG. 11A-11B). However, the expression of R475C-DDX3X failed to rescue 5′-UTR unwinding (FIG. 11A-11B), thus confirming that R475C substitution resulted in loss of helicase activity of the DDX3X protein.

### Example 3: DDX3X Somatic Mutants Identified in a Subgroup of Lymphoma Patients Exhibit Loss of Helicase Function

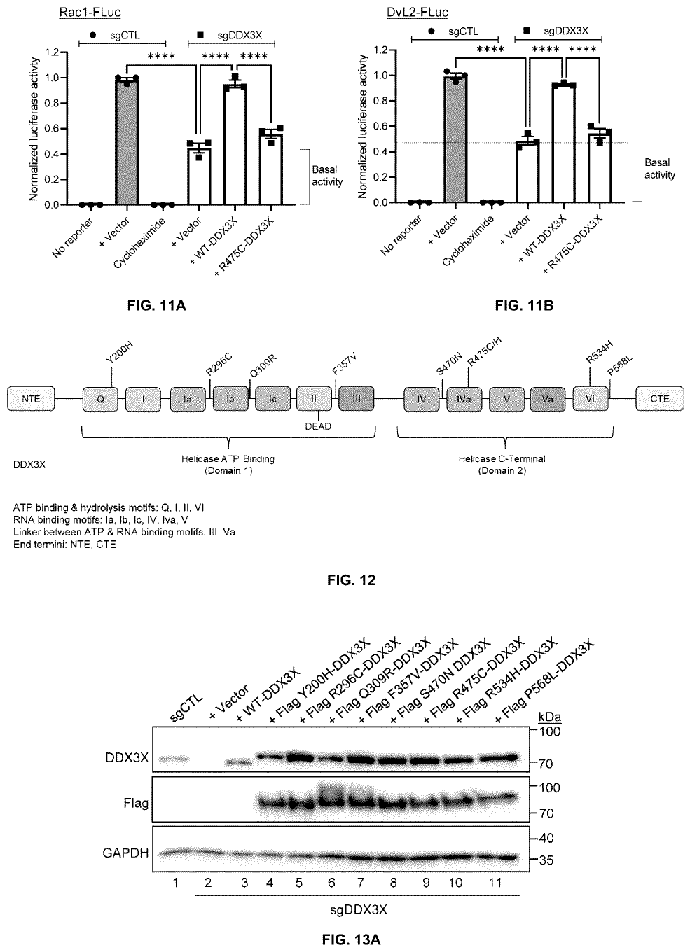

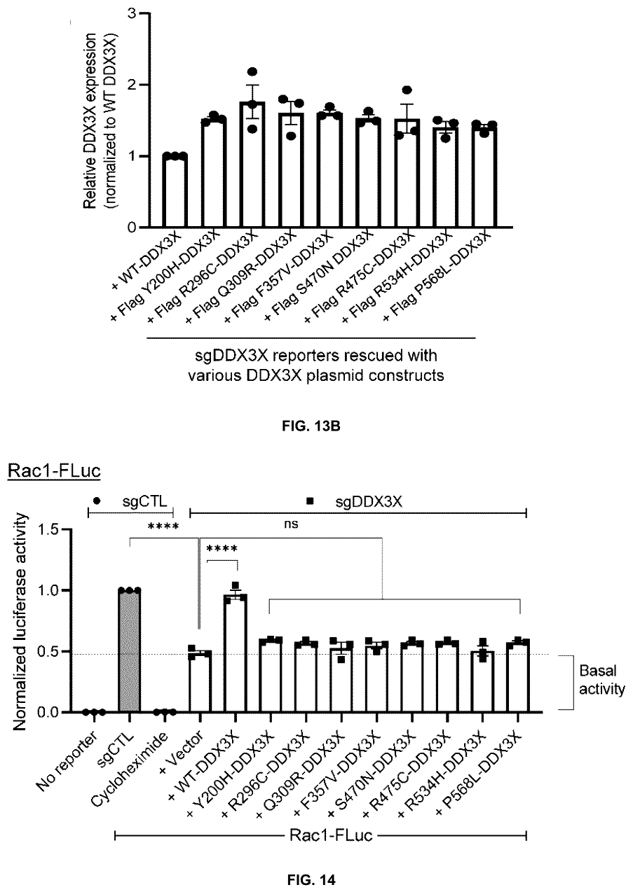

Earlier, eight different somatic point mutations in the DDX3X gene (FIG. 12) were identified in a cohort of relapsed/refractory DLBCL patients [8]. PolyPhen-2 bioinformatic analysis predicted these mutations to be “damaging [8]. To confirm whether these mutations cause loss of helicase activity in cells, plasmid vectors were generated containing each of the eight DDX3X mutations and expressed them in the sgDDX3X Rac1-Fluc 293T reporter cells FIG. 13A, lanes 4-11). Luciferase assays showed that none of the eight DDX3X mutants could unwind the target 5′-UTR mRNA as effectively as WT-DDX3X (FIG. 14).

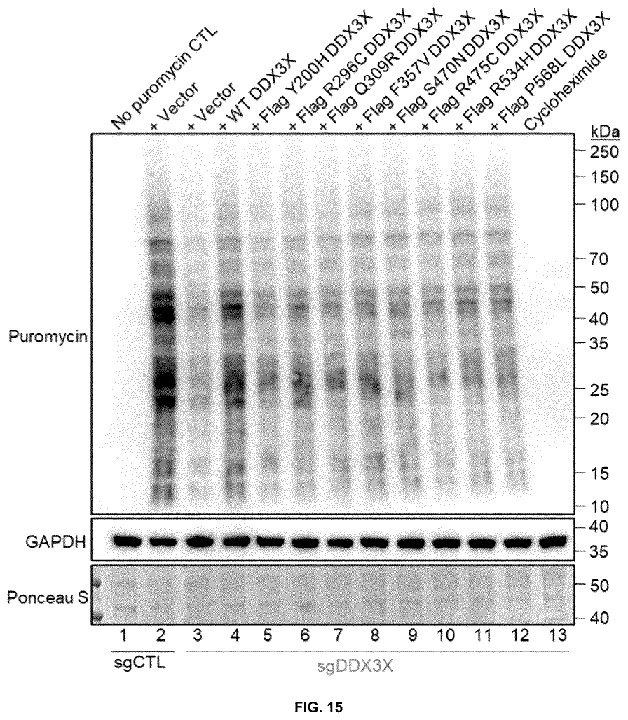

Since DDX3X is involved in the pre-assembly of translation-initiation complexes to facilitate cap-dependent translation via its helicase activity all above transgenic cells were subjected to a puromycin incorporation assay that measures global protein synthesis. Results showed that protein synthesis in cells expressing the DDX3X mutant plasmids was significantly impaired (FIG. 15, lanes 5-11). Of note, DDX3X mutations at the three residues R475C/G, P568L, and R534H have previously been shown to have defective helicase activity [15,16]. Since all eight DDX3X mutations have been found to be associated with poor prognosis in DLBCL patients [8] it can be suggested that inability of DDX3X to unwind RNA plays either a direct or an indirect role in DLBCL progression and/or chemoresistance.

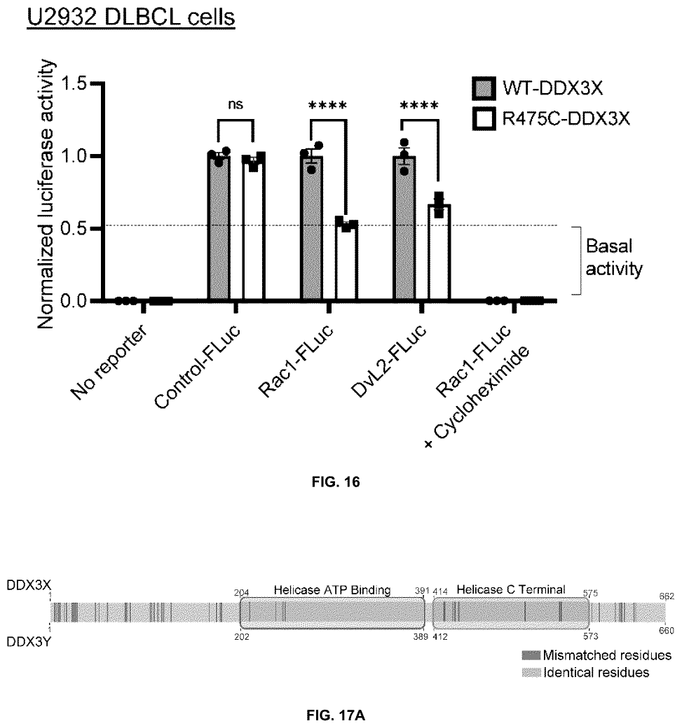

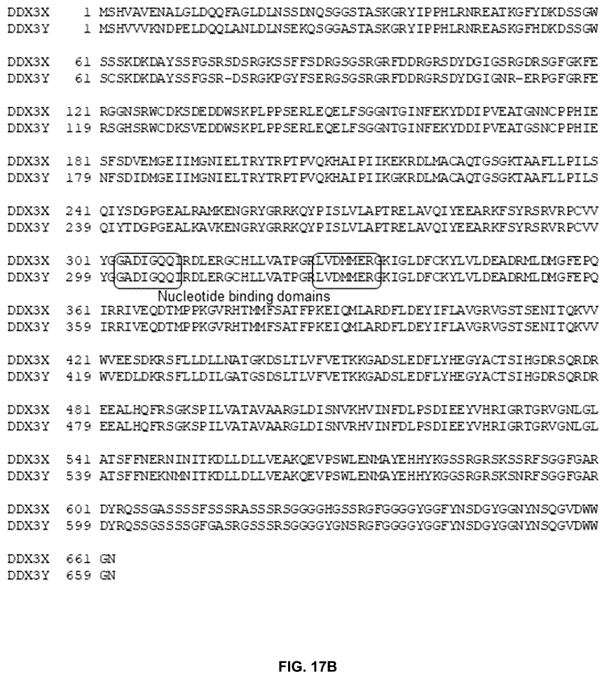

To further substantiate the helicase-dead phenotype of mutant DDX3X in non-Hodgkin lymphoma patients, the DDX3X gene engineering approach was extended to the U2932 cell line that was established from ascites in a patient suffering from DLBCL [17].For this, a point mutation at the R475C residue in the DDX3X gene (DDX3X-R475C) in U2932 cells was first generated using the CRISPR/Cas9 knock-in technique, as described [16]. Subsequently, the reporter plasmid Rac1-Fluc or DvL2-Fluc was expressed in R475C-DDX3X U2932 cells and luciferase signal was recorded. In comparison to U2932 cells with WT DDX3X, cells containing R475C-DDX3X mutation exhibited significantly impaired helicase unwinding activity (FIG. 16).

### Example 4: ICD-Helicase Cell Systems Reveal Subtle Differences Between DDX3X and DDX3Y Helicase Functions

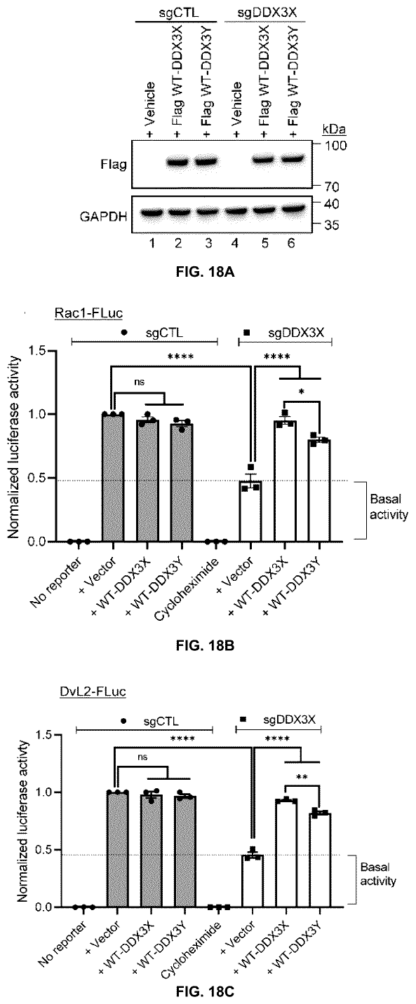

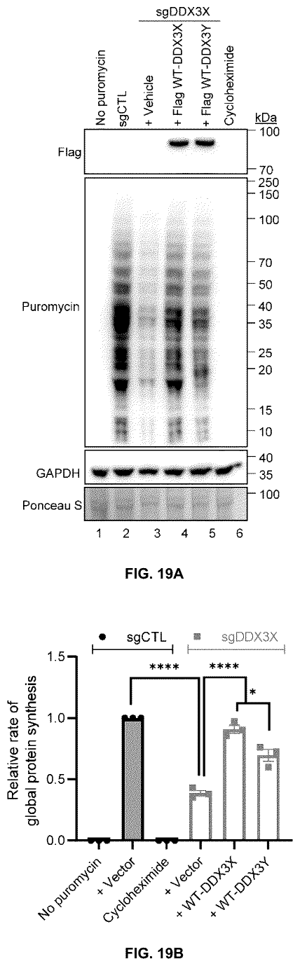

To further define the sensitivity of the developed ICD-Helicase reporter system, the usage of this tool was extended to interrogate the poorly characterized Y chromosome homolog, DDX3Y. DDX3X and DDX3Y share functional homology [18, 19] with intrinsically disordered regions (IDR) in each of the N and C termini of the non-helicase regions (FIG. 17A). Sequence alignment (FIG. 17B) revealed a conserved nucleotide binding domain necessary for helicase activity indicating that DDX3Y may interact with ATP and double stranded RNA in a manner similar to DDX3X. Since DDX3Y can compensate for the loss of DDX3X function [16], WT DDX3X or WT DDX3Y was expressed in the 293T reporter cell system to confirm this observation (FIG. 18A). In control sgCTL cells, overexpression of either WT-DDX3X or WT-DDX3Y did not cause any significant change in the luminescence signal, in both Rac1-FLuc and DvL2-FLuc 5′UTRs (FIG. 18B, FIG. 18C, probably due to a saturation in DDX3 helicase unwinding activities by endogenous proteins. Exogenous expression of WT-DDX3X or WT-DDX3Y in sgDDX3X 293T cells (cells without endogenous DDX3X and DDX3Y) could rescue the helicase unwinding activity of cells (FIG. 18B. FIG. 18C). Similarly, exogenous expression of WT-DDX3X or WT-DDX3Y in sgDDX3X 293T cells could rescue the ability of cells to translate proteins (FIG. 19A, lanes 4 and 5 vs lane 3). It was also noted that DDX3Y helicase is less efficient in RNA unwinding as well as protein translation compared to DDX3X (FIG. 19B), suggesting subtle functional differences despite both proteins sharing >90% sequence homologies.

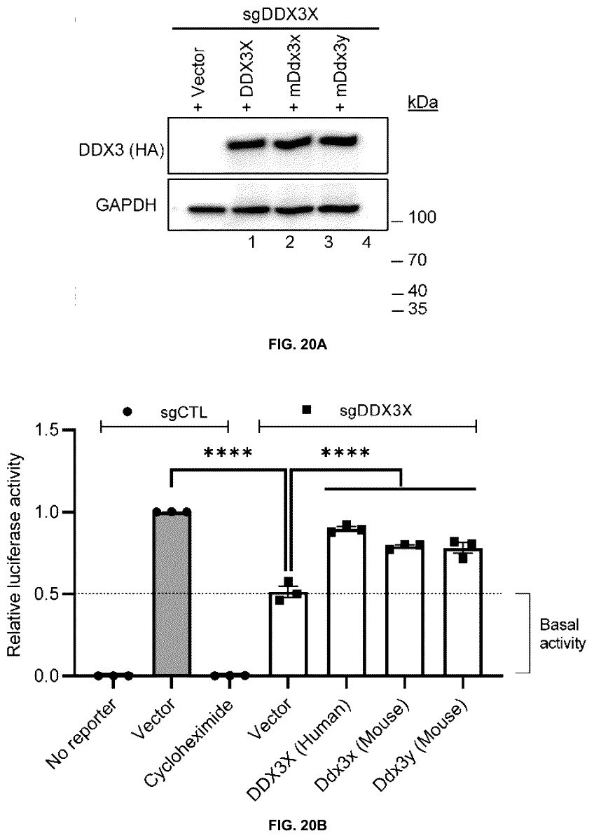

Next, the utility of the reporter cell system was extended to quantify mouse Ddx3 (mDdx3) helicase activity. In contrast to the two homologous genes (DDX3X & DDX3Y) present in humans, there are three homologues DDX3 genes (mDdx3x, mDdx3y & D1Pas1-PL10) in mice [20,21]. Both mouse Ddx3x and Ddx3y are ubiquitously expressed throughout the mouse with >99% homology with human DDX3X and DDX3Y, suggesting conserves function between species [5]. However, D1Pas1-PL10 is expressed only in mouse sperm [5]. Exogenous expression of mouse mDdx3× or mDdx3y (FIG. 20A) in sgDDX3X 293T cells could rescue the helicase unwinding activity of cells similar to the levels of human WT-DDX3X (FIG. 20B). Collectively, these data demonstrate the versatility of the reporter system beyond human DDX3 paralogs.

### Example 5: Both Helicase and ATPase Activities are Necessary for DDX3Y Function

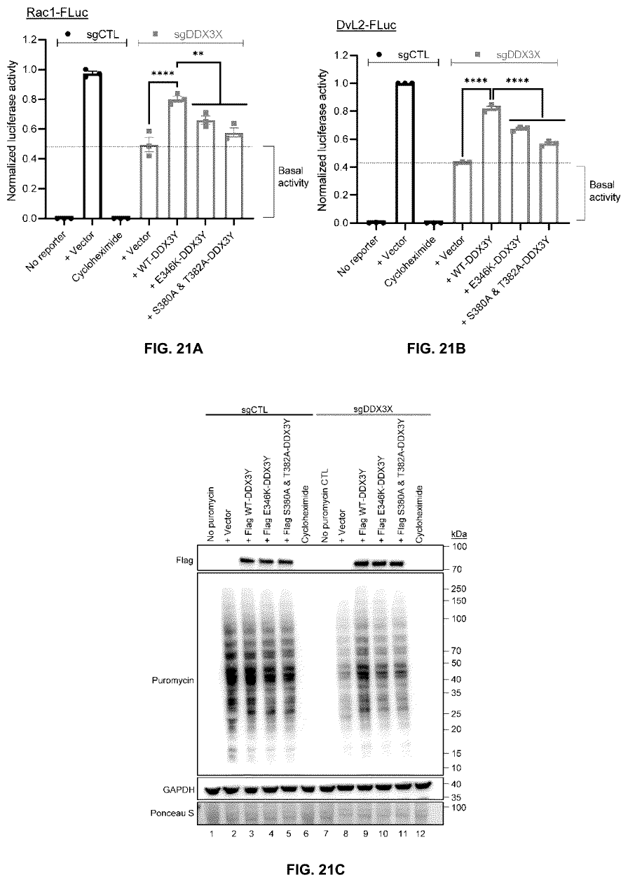

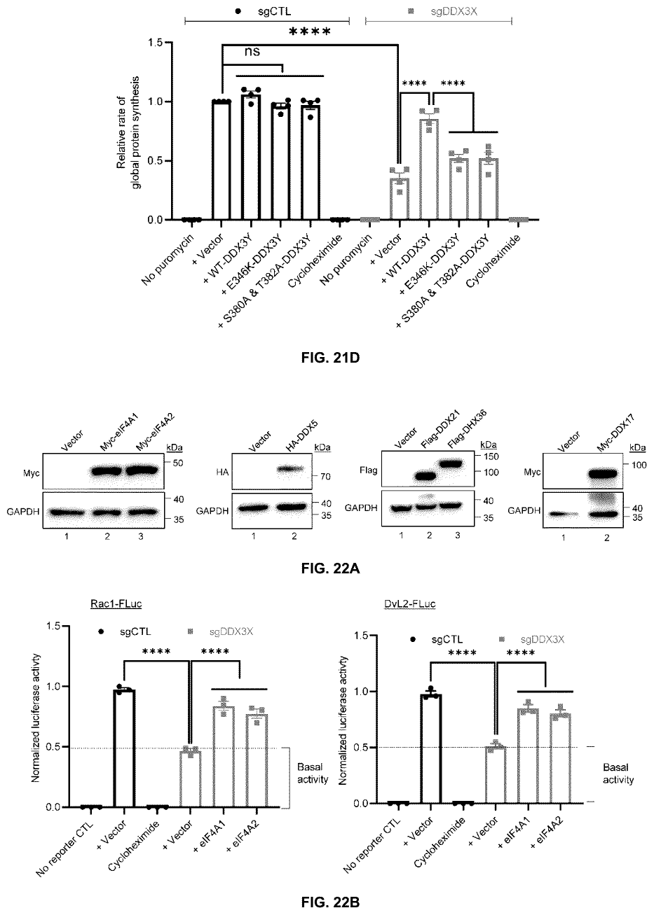

Developing specific inhibitors against either of the DDX3 homologues (DDX3X and DDX3Y) requires an in depth understanding of their homologue-specific functions. Based on sequence homologies of DDX3X and DDX3Y, 2 defective mutants were generated-1) DDX3Y E346K harbouring a point-mutation in the catalytic DEAD-box domain that impairs its helicase and ATPase activity and 2) DDX3Y S380A & T382A harbouring two point-mutations exhibiting defective helicase activity but retaining the ATPase activity of the helicase. Both mutants were expressed in the DDX3 reporter 293T cells and found that both mutations impaired the ability of DDX3Y to rescue cellular luciferase activity, in both in Rac1-FLuc and DvL2-FLuc (FIG. 21A, FIG. 21B. Similar results were obtained in terms of their inability to rescue global protein synthesis (FIG. 21C, lanes 10 & 11 vs lane 9). A modest increase in protein synthesis as compared to control vector (FIG. 21C, lane 8 vs lanes 10 & 11) indicates that the non-helicase domains of DDX3Y may be involved in translation regulation through direct or indirect interaction with mRNAs. These suggest that, for designing specific DDX3Y inhibitors, one must consider targeting beyond just the helicase region to fully disrupt DDX3Y functions.

### Example 6: Rationale of Selecting 5′ UTRs

To develop the DDX3X reporter cells, a recent paper Calviello et al [12]., showed a total of five 5′ UTRs that are sensitive to DDX3 depletion in HEK 293T cells. Rac1 and DvL2 5′-UTRs were selected as they were the most sensitive to DDX3 depletion. Subsequently, plasmids were designed using the Rac1 and DvL2 5′-UTR sequences and that were inserted upstream of the firefly luciferase. These reporter plasmids were then custom synthesized from VectorBuilder. VB220922-1486nnk (pcDNA3.1 (+)-5′-UTR_RAC1: luciferase) and VB220922-1483jcb (pcDNA3.1 (+)-5′-UTR_DVL2: luciferase). FIG. 5 shows that sgDDX3X 293T cells (i.e., DDX3X knockout cells) exhibit defective helicase activity (luciferase signal) in Rac1-Fluc and DvL2-Fluc, but not control in FLuc (plasmid without 5′-UTR). This result provided the basis for using the two reporter cells to monitor the helicase activity of DDX3X in cell culture.

To study potential translational regulation of DDX3Y as a possible reason for its expression in prostate cancer, DDX3Y 5′-UTR were cloned from the gDNA of human primary peripheral blood mononuclear cells (PBMCs; sequence identified from NCBI). Overlap PCR was then performed with FLuc open reading frame before cloning into pIRES2-AcGFP1 (TakaraBio #632435) to generate DDX3Y 5′-UTR reporter construct. This construct was not commercially obtained. This construct can be used as another example to demonstrate the specificity of DDX3X in regulating specific 5′-UTRs. FIG. 16B, FIG. 16C show that sgDDX3X 293T cells exhibit defective helicase activity (luciferase signal) in Rac1-Fluc and DvL2-Fluc, but not in cells expressing DDX3Y, supporting the specificity of only a subset of 5′-UTRs affected by DDX3X depletion.

### Example 7: Optimize Cell Treatment Conditions for the Luciferase Assay

The ICD-Helicase reporter system can be fine-tunned by incorporating Aequorea coerulescens GFP (AcGFP) as an internal control that allowed us to normalize the possible variations in transfection efficiency between experiments (FIG. 1B-1E). Further, in the new improved reporter plasmid, an internal ribosome entry site (IRES) was inserted between the AcGFP and the firefly luciferase (FLuc) to allow independent expression through a cap independent translation (FIG. 1B-1E). These modifications improved the ICD-Helicase reporter system and the measurements of helicase activity using both Rac1 5-′UTR as well as DvL2 5′-UTR.

The improved version of the ICD-Helicase reporter system was examined in 293T cells with endogenous DDX3X (sgCTL) as wells in cells with depleted DDX3X (sgDDX3X). Data showed that DDX3X-depleted sgDDX3X 293T cells exhibited significantly reduced luciferase signals in both Rac1-FLuc and DVL2-Fluc reporters with respective 5′-UTRs, but no in cells without 5′-UTR (Control-FLuc) (FIG. 5).

The ICD-Helicase reporter system was validated using a known DDX3 inhibitor RK-33. 293T cells with normal endogenous DDX3 (sgCTL) displayed a dose-dependent reduction in luciferase activity upon chemical inhibition of DDX3. However, in the absence of the DDX3 protein, sgDDX3X 293T cells expressing either Rac1-FLuc or DvL2-FLuc construct did not respond to RK-33 (FIG. 3, FIG. 8). These demonstrate the ability of our ICD-Helicase reporter system to be applicable to screen for DDX3X and DDX3Y specific inhibitors.

Notably, DDX3X-depleted reporter cells exhibited a basal level of luciferase signals. This could be due to other members of the DEAD box helicase family proteins present in sgDDX3X 293T cells that can also partially unwind the complex 5′UTR. To investigate this possibility, five closely-related DEAD-box helicases elF4A1, elF4A2, DDX5, DDX21, and DDX17 as well as the DEAH-box helicase DHX36 [selected based on the similarity with DDX323] were exogenously expressed in sgDDX3X reporter cells (FIG. 22A). Increased luciferase signals were noted in both Rac1-FLuc and DvL2-FLuc 5′UTRs due to either of the six DEAD/DEAH-box helicases (FIG. 22B, FIG. 22C). Collectively, these results established Rac1-FLuc and DvL2-Fluc reporters to be sensitive to DDX3X alteration.

Next, the improved ICD-Helicase reporter system was applied to determine the helicase activity of the R475C-DDX3X mutation (identified and reported in [8]) and compared that with WT-DDX3X. For this, WT-DDX3X or mutant R475C-DDX3X plasmids were exogenously expressed in sgDDX3X 293T cells (FIG. 9) and confirmed their expression by Western immunoblotting (FIG. 10A). Reconstituting sgDDX3X 293T cells with WT-DDX3X rescued the ability of cells to unwind the target 5′-UTRs, while expression of R475C-DDX3X failed to unwind the target 5′-UTRs (FIG. 11A, FIG. 11B. These results demonstrated loss of helicase activity of the mutant R475C-DDX3X protein using the improved ICD-Helicase reporter system.

These experiments were able to be performed in 24-well plates. To further develop the approach to be applicable to high throughput setting, sgCTL and sgDDX3X 293T cells were generated that stably express the bi-cistronic reporter as depicted in FIG. 1A-1E. These stable reporter cells can be used in a 96-well or 386-well format that will reduce the use of reagents and resources and can be better fit for a cost-effective product development in future.

### Example 8: Validation of Optimized Cell Treatment Conditions for the Luciferase Assay

To validate the reporter system in multiple cell lines, the ICD-Helicase reporter plasmids were expressed in Diffuse Large B-Cell Lymphoma (DLBCL) cell line U2932, Prostate Epithelial cell line RWPE-1, and Metastatic Prostate Carcinoma cell line LNCaP. To substantiate the helicase-dead phenotype of mutant DDX3X in non-Hodgkin lymphoma patients, the DDX3X gene engineering approach was extended to the U2932 cell line that was established from ascites in a patient suffering from DLBCL. For this, a point mutation at the R475C residue was first generated in the DDX3X gene (DDX3X-R475C) in U2932 cells using the CRISPR/Cas9 knock-in technique [8]. Subsequently, reporter plasmids were expressed in wild-type and R475C mutant U2932 cells and luciferase signals were recorded. In comparison to U2932 cells with WT-DDX3X, cells containing R475C-DDX3X mutation exhibited significantly impaired helicase unwinding activity with Rac1-FLuc and DvL2-FLuc (FIG. 16).

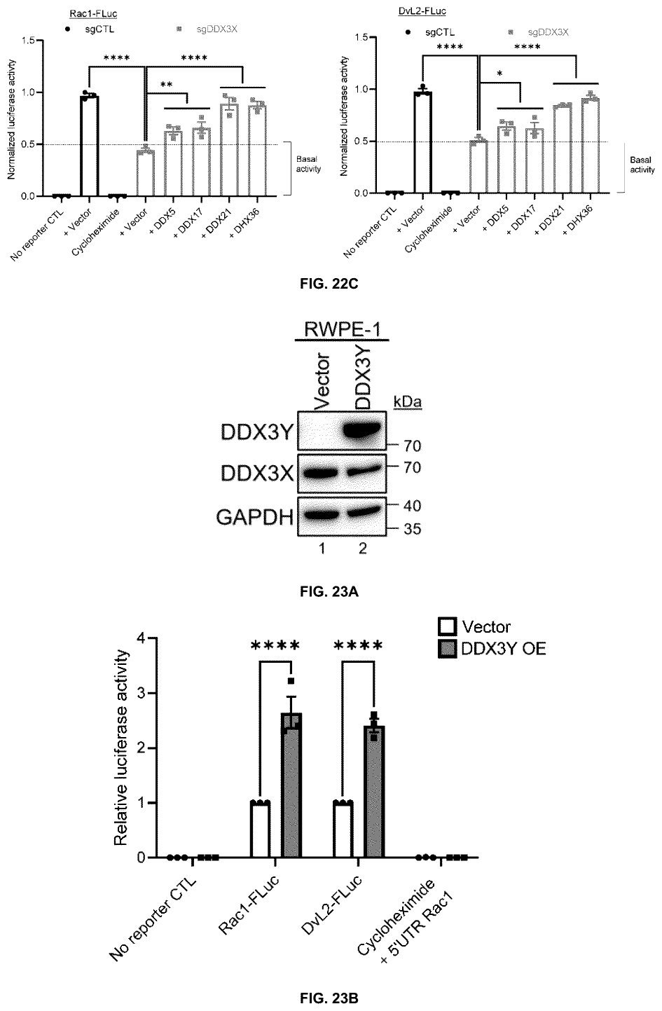

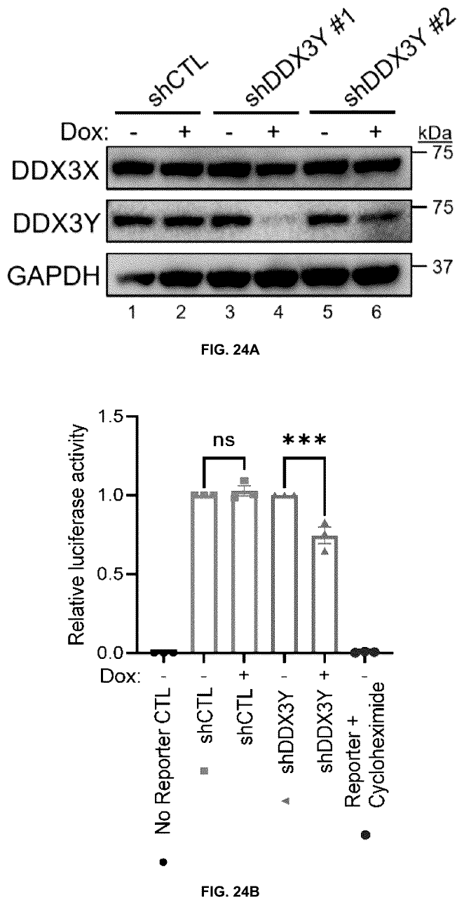

RWPE-1 cells do not express the DDX3Y paralog. Therefore, DDX3Y was genetically overexpressed in RWPE-1 cells (FIG. 23A) and transiently expressed the improved ICD-Helicase reporter plasmids in these cells. DDX3Y overexpressing cells exhibited enhanced luciferase signal in Rac1-FLuc and DvL2-FLuc reporter system indicative of increased helicase activity (FIG. 23B).

In LNCaP cells, a doxycycline-inducible DDX3Y knockdown system was stably expressed where addition of doxycycline into in-vitro cultures specifically depleted DDX3Y in the cells without impacting the expression of DDX3X (FIG. 24A). Next, the Rac1-FLuc reporter plasmid system was expressed into control (shCTL) and DDX3Y-depleted (shDDX3Y) LNCaP cells. DDX3Y knockdown in LNCaP cells significantly reduced helicase activity in shDDX3Y cells (FIG. 24B). These results further validated that the improved ICD-Helicase reporter system works well in multiple cell types.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The cells, systems, methods, kits and uses described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

### REFERENCES

1. Linder, P., and Jankowsky, E. (2011). From unwinding to clamping—the DEAD box RNA helicase family. Nat. Rev. Mol. Cell. Biol. 12, 505-516.
2. Kim, Y. S., Lee, S. G., Park, S. H., and Song, K. (2001). Gene structure of the human DDX3 and chromosome mapping of its related sequences. Mol. Cells 12, 209-214.
3. Ditton, H. J., Zimmer, J., Kamp, C., Rajpert-De Meyts, E., and Vogt, P. H. (2004). The AZFa gene DBY (DDX3Y) is widely transcribed but the protein is limited to the male germ cells by translation control. Hum. Mol. Genet. 13, 2333-2141.
4. Mo, J., Liang, H., Su, C., Li, P., Chen, J., and Zhang, B. (2021). DDX3X: structure, physiologic functions and cancer. Mol. Cancer 20, 38.
5. Li, Q., Zhang, P., Zhang, C., Wang, Y., Wan, R., Yang, Y., Guo, X., Huo, R., Lin, M., Zhou, Z., et al. (2014). DDX3X regulates cell survival and cell cycle during mouse early embryonic development. J. Biomed. Res. 28, 282-291.
6. Snijders Blok, L., Madsen, E., Juusola, J., Gilissen, C., Baralle, D., Reijnders, M. R., Venselaar, H., Helsmoortel, C., Cho, M. T., Hoischen, A., et al. (2015). Mutations in DDX3X are a common cause of unexplained intellectual disability with gender-specific effects on Wnt signaling. Am. J. Hum. Genet. 97, 343-352.
7. Lin, T. C. (2019). DDX3X multifunctionally modulates tumour progression and serves as a prognostic indicator to predict cancer outcomes. Int. J. Mol. Sci. 21, 281.
8. Kizhakeyil, A., Zaini, N. B. M., Poh, Z. S., Wong, B. H. S., Loh, X., Ng, A. S., Low, Z. S., Prasannan, P., Gong, C., Tan, M. G. K., et al. (2021). DDX3X loss is an adverse prognostic marker in diffuse large B-cell lymphoma and is associated with chemoresistance in aggressive non-Hodgkin lymphoma subtypes. Mol. Cancer 20, 134.
9. Jiang, L., Gu, Z. H., Yan, Z. X., Zhao, X., Xie, Y. Y., Zhang, Z. G., Pan, C. M., Hu, Y., Cai, C. P., Dong, Y., et al. (2015). Exome sequencing identifies somatic mutations of DDX3X in natural killer/T-cell lymphoma. Nat. Genet. 47, 1061-1066.
10. Oh, S., Flynn, R. A., Floor, S. N., Purzner, J., Martin, L., Do, B. T., Schubert, S., Vaka, D., Morrissy, S., Li, Y., et al. (2016). Medulloblastoma-associated DDX3 variant selectively alters the translational response to stress. Oncotarget 7, 28169-28182.
11. Lim, J. Q., Huang, D., Chan, J. Y., Laurensia, Y., Wong, E. K. Y., Cheah, D. M. Z., Chia, B. K. H., Chuang, W. Y., Kuo, M. C., Su, Y. J., et al. (2022). A genomic-augmented multivariate prognostic model for the survival of natural-killer/T-cell lymphoma patients from an international cohort. Am. J. Hematol. 97, 1159-1169.
12. Calviello, L., Venkataramanan, S., Rogowski, K. J., Wyler, E., Wilkins, K., Tejura, M., Thai, B., Krol, J., Filipowicz, W., Landthaler, M., and Floor, S. N. (2021). DDX3 depletion represses translation of mRNAs with complex 5′ UTRs. Nucleic Acids Res. 49, 5336-5350.
13. Yang, S. N. Y., Atkinson, S. C., Audsley, M. D., Heaton, S. M., Jans, D. A., and Borg, N. A. (2020). RK-33 is a broad-spectrum antiviral agent that targets DEAD-Box RNA helicase DDX3X. Cells 9, 170.
14. Lee, C. S., Dias, A. P., Jedrychowski, M., Patel, A. H., 391 Hsu, J. L., and Reed, R. (2008). Human DDX3 functions in translation and interacts with the translation initiation factor elF3. Nucleic Acids Res. 36, 4708-4718.
15. Chapuy, B., Stewart, C., Dunford, A. J., Kim, J., Kamburov, A., Redd, R. A., Lawrence, M. S., Roemer, M. G. M., Li, A. J., Ziepert, M., et al. (2018). Molecular subtypes of diffuse large B cell lymphoma are associated with distinct pathogenic mechanisms and outcomes. Nat. Med. 24, 679-690.
16. Gong, C., Krupka, J. A., Gao, J., Grigoropoulos, N. F., Giotopoulos, G., Asby, R., Screen, M., Usheva, Z., Cucco, F., Barrans, S., et al. (2021). Sequential inverse dysregulation of the RNA helicases DDX3X and DDX3Y facilitates MYC-driven lymphomagenesis. Mol. Cell 81, 4059-4075.e11.
17. Amini, R. M., Berglund, M., Rosenquist, R., Von Heideman, A., Lagercrantz, S., Thunberg, U., Bergh, J., Sundström, C., Glimelius, B., and Enblad, G. (2002). A novel B-cell line (U-2932) established from a patient with diffuse large B-cell lymphoma following Hodgkin lymphoma. Leuk. Lymphoma 43, 2179-2189.
18. Venkataramanan, S., Gadek, M., Calviello, L., Wilkins, K., and Floor, S. N. (2021). DDX3X and DDX3Y are redundant in protein synthesis. RNA 27, 1577-1588.
19. Shen, H., Yanas, A., Owens, M. C., Zhang, C., Fritsch, C., Fare, C. M., Copley, K. E., Shorter, J., Goldman, Y. E., and Liu, K. F. (2022). Sexually dimorphic RNA helicases DDX3X and DDX3Y differentially regulate RNA metabolism through phase separation. Mol. Cell 82, 2588-2603.e9.
20. Sekiguchi, T., lida, H., Fukumura, J., and Nishimoto, T. (2004). Human DDX3Y, the Y413 encoded isoform of RNA helicase DDX3, rescues a hamster temperature-sensitive ET24 mutant cell line with a DDX3X mutation. Exp. Cell Res. 300, 213-222.
21. Vong, Q. P., Li, Y., Lau, Y. F., Dym, M., Rennert, O. M., and Chan, W. Y. (2006). Structural characterization and expression studies of Dby and its homologs in the mouse. J. Androl. 27, 653-661.

1. A reporter cell for assessing the activity of a DDX3 helicase, comprising:
a circular DNA molecule comprising a reporter expression construct comprising:
a reporter gene operably linked to one or more regulatory elements capable of controlling expression of the reporter gene; and
a nucleotide sequence encoding a 5′ untranslated region (5′-UTR) positioned upstream of the reporter gene, wherein a secondary structure of the 5′-UTR is capable of being unwound by a functionally active DDX3 helicase to facilitate the expression of the reporter gene and emission of a detectable signal,
wherein the reporter cell lacks endogenous expression of the DDX3 helicase.

2. The reporter cell of claim 1, wherein the DDX3 helicase is a human DDX3X or DDX3Y helicase, or variants thereof.

3. The reporter cell of claim 1, wherein the reporter cell naturally or artificially lacks endogenous expression of the DDX3 helicase, more preferably the reporter cell is a DDX3 knock-out reporter cell.

4. The reporter cell of claim 1, further comprising a second circular DNA molecule comprising a nucleotide sequence encoding the DDX3 helicase, and one or more regulatory elements capable of controlling exogenous or transient expression of the DDX3 helicase, preferably the second circular DNA molecule is an expression plasmid for transient and/or exogenous expression of the DDX3 helicase.

5. The reporter cell of claim 1, wherein the reporter gene is a luminescent reporter gene, preferably a gene encoding firefly luciferase (FLuc), a gene encoding renilla luciferase (Rluc), a gene encoding Cypridina Luciferase (Cluc), a gene encoding Gaussia Luciferase (Gluc), or a gene encoding NanoLuc Luciferase (NanoLuc), more preferably the reporter gene is a gene encoding firefly luciferase (FLuc).

6. The reporter cell of claim 1, wherein the nucleotide sequence encoding the 5′-UTR may be derived from a 5′-UTR of the RAC1 or DVL2 gene, preferably the nucleotide sequence encoding the 5′-UTR comprises or consists of a nucleotide sequence as set forth in any one of SEQ ID NO: 14 or 17 or variants thereof.

7. The reporter cell of claim 1, wherein the reporter expression construct further comprises an Internal Ribosome Entry Site (IRES) and a fluorescent marker gene, wherein the IRES is positioned downstream of the reporter gene, and the fluorescent marker gene is positioned downstream of the IRES.

8. The reporter cell of claim 1, wherein the reporter cell is a mammalian cell derived from a mammalian subject or a mammalian cell line, preferably the mammalian cell is a primary cell derived from a human subject or a human cell derived from a human cell line selected from Hela cells and human embryonic kidney (HEK-293) cells, diffuse large B-cell lymphoma cells (U2932), prostate epithelial cells (RWPE-1), Metastatic prostate carcinoma cells (LNCaP), more preferably the reporter cell is a human cell derived from an embryonic kidney 293 (HEK 293) cell line or derivatives thereof.

9. A method for assessing the activity of a DDX3 helicase, comprising:
providing a reporter cell according to claim 1;
culturing the reporter cell under conditions that allow expression of the reporter gene to produce a reporter gene product, wherein the DDX3 helicase is exogenously or transiently expressed in the reporter cell; and
detecting a presence/absence, optionally quantity, of a detectable signal emitted by the reporter gene product to obtain a readout that is indicative of the activity of the DDX3 helicase.

10. The method of claim 9, wherein a presence, optional quantity, of the detectable signal emitted by the reporter gene product indicates that the exogenously or transiently expressed DDX3 helicase is functionally active and has unwound a secondary structure of the 5′-UTR facilitating expression of the reporter gene and emission of the detectable signal.

11. The method of claim 9, wherein the reporter gene is a luciferase reporter gene, and the detectable signal is a luminescence signal, and wherein a quantitative readout of the luminescence signal is proportional to the expression level of the luciferase reporter gene and correlates to the activity of the DDX3 helicase.

12. A method for developing or identifying an agent capable of modulating the activity of DDX3 helicase, the method comprising:
providing a reporter cell according to claim 1, wherein the reporter cell exogenously or transiently expresses a functionally active DDX3 helicase;
administering a candidate agent to the reporter cell;
measuring a detectable signal emitted by a reporter gene product;
comparing the measured signal to a control,
wherein the candidate agent is identified as capable of modulating the activity of DDX3 helicase, if the measured detectable signal of the reporter gene product has increased or decreased relative to the control.

13. The method of claim 12, wherein a decrease of the measured detectable signal of the reporter gene product relative to the control is indicative of the candidate agent having inhibitory activity of DDX3 helicase, or an increase of the measured detectable signal of the reporter gene product relative to the control is indicative of the candidate agent having stimulatory activity of DDX3 helicase.
